SEQUENCE LISTING (56 sequences)

SEQ ID NO: 1 - protein, 17 aa, Homo sapiens
SPQLATLADEVSASLAK

SEQ ID NO: 2 - protein, 115 aa, Artificial Sequence
  hTau60 VH
EVQLQQSGPELVKPGTSVKISCKVFGYTFTDYYMNWVKQSHGKSLEWIGDINPDNGETTYNQKFKGKATLTVDKSSSTAYMELRSLTSEDSAVYYCAKGATFVYWGQGTLVTVSA

SEQ ID NO: 3 - protein, 112 aa, Artificial Sequence
  hTau60 VL
DVVMTQTPLSLPVSLGDQASISCISSQSLVHSTGTTFLHWFLQKPGQSPKLLIYKVSNRFSGVPDRFSGSGSGTDFTLKISRVEAEDLGFYFCSQSTYFPLTFGSGTKLEIK

SEQ ID NO: 4 - protein, 10 aa, Artificial Sequence
  hTau60 HCDR1
GYTFTDYYMN

SEQ ID NO: 5 - protein, 17 aa, Artificial Sequence
  hTau60 HCDR2
DINPDNGETTYNQKFKG

SEQ ID NO: 6 - protein, 6 aa, Artificial Sequence
  hTau60 HCDR3
GATFVY

SEQ ID NO: 7 - protein, 16 aa, Artificial Sequence
  hTau60 LCDR1
ISSQSLVHSTGTTFLH

SEQ ID NO: 8 - protein, 7 aa, Artificial Sequence
  hTau60 LCDR2
KVSNRFS

SEQ ID NO: 9 - protein, 9 aa, Artificial Sequence
  hTau60 LCDR3
SQSTYFPLT

SEQ ID NO: 10 - protein, 445 aa, Artificial Sequence
  hTau60 HC
EVQLQQSGPELVKPGTSVKISCKVFGYTFTDYYMNWVKQSHGKSLEWIGDINPDNGETTYNQKFKGKATLTVDKSSSTAYMELRSLTSEDSAVYYCAKGATFVYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 11 - protein, 219 aa, Artificial Sequence
  hTau60 LC
DVVMTQTPLSLPVSLGDQASISCISSQSLVHSTGTTFLHWFLQKPGQSPKLLIYKVSNRFSGVPDRFSGSGSGTDFTLKISRVEAEDLGFYFCSQSTYFPLTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 115 aa, Artificial Sequence
  PT/66 VH
QVQLQQPGAELVKPGASVKMSCKASGYTFTSYWITWVKQRPGQGLEWIGDIHPGRGSTKSNEKFKSKATLTVDTSSSTAYMQFSSLTSEDSAVYYCARRWGFDYWGQGTTLTVSS

SEQ ID NO: 13 - protein, 112 aa, Artificial Sequence
  PT/66 VL
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVDYPLTFGAGTKLELK

SEQ ID NO: 14 - protein, 10 aa, Artificial Sequence
  PT/66 HCDR1
GYTFTSYWIT

SEQ ID NO: 15 - protein, 17 aa, Artificial Sequence
  PT/66 HCDR2
DIHPGRGSTKSNEKFKS

SEQ ID NO: 16 - protein, 6 aa, Artificial Sequence
  PT/66 HCDR3
RWGFDY

SEQ ID NO: 17 - protein, 16 aa, Artificial Sequence
  PT/66 LCDR1
RSSKSLLYKDGKTYLN

SEQ ID NO: 18 - protein, 7 aa, Artificial Sequence
  PT/66 LCDR2
LMSTRAS

SEQ ID NO: 19 - protein, 9 aa, Artificial Sequence
  PT/66 LCDR3
QQLVDYPLT

SEQ ID NO: 20 - protein, 445 aa, Artificial Sequence
  PT/66 HC
QVQLQQPGAELVKPGASVKMSCKASGYTFTSYWITWVKQRPGQGLEWIGDIHPGRGSTKSNEKFKSKATLTVDTSSSTAYMQFSSLTSEDSAVYYCARRWGFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 21 - protein, 219 aa, Artificial Sequence
  PT/66 LC
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVDYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 22 - protein, 115 aa, Artificial Sequence
  PT/69 VH
QVQLQQPGAELVKPGASVKMSCKASGYTFTNYWITWVKQRPGQGLEWIGDIYPGSGRTKSNEKFKNKATLTADTSSSTAYMQLSSLTSEDSAVYYCTRRWGLDYWGQGTTLTVSS

SEQ ID NO: 23 - protein, 112 aa, Artificial Sequence
  PT/69 VL
DIVITQDELSNPVTSGESVSISCRSNKSLLYKDGKTYLNWFLQRPGQSPQLLIYLMSTRASGVSDRFSGSGSGTDFTLEISRVKAEDVGVYYCQQLVDYPLTFGAGTKLELK

SEQ ID NO: 24 - protein, 10 aa, Artificial Sequence
  PT/69 HCDR1
GYTFTNYWIT

SEQ ID NO: 25 - protein, 17 aa, Artificial Sequence
  PT/69 HCDR2
DIYPGSGRTKSNEKFKN

SEQ ID NO: 26 - protein, 6 aa, Artificial Sequence
  PT/69 HCDR3
RWGLDY

SEQ ID NO: 27 - protein, 16 aa, Artificial Sequence
  PT/69 LCDR1
RSNKSLLYKDGKTYLN

SEQ ID NO: 28 - protein, 445 aa, Artificial Sequence
  PT/69 HC
QVQLQQPGAELVKPGASVKMSCKASGYTFTNYWITWVKQRPGQGLEWIGDIYPGSGRTKSNEKFKNKATLTADTSSSTAYMQLSSLTSEDSAVYYCTRRWGLDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 29 - protein, 219 aa, Artificial Sequence
  PT/69 LC
DIVITQDELSNPVTSGESVSISCRSNKSLLYKDGKTYLNWFLQRPGQSPQLLIYLMSTRASGVSDRFSGSGSGTDFTLEISRVKAEDVGVYYCQQLVDYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 30 - protein, 117 aa, Artificial Sequence
  PT/53 VH
EVKLMESGGGLVQPGASLRLSCAASGFTFTDYYMSWVRQPPGKAPEWLALIRNKANGYTTKYAASVKGRFTISRDNSQNILYLQMNTLRAEDSATYYCVKAVWFAYWGQGTLVTVSA

SEQ ID NO: 31 - protein, 112 aa, Artificial Sequence
  PT/53 VL
DIVITQDELSNPVTSGESVSISCRSSKSLLYKDGKTYLNWFLQRPGQSPQLLIYLMSTRASGVSDRFSGSGSGTDFTLEISRVKAEDVGVYYCLQLVEYPYTFGGGTKLEIK

SEQ ID NO: 32 - protein, 10 aa, Artificial Sequence
  PT/53 HCDR1
GFTFTDYYMS

SEQ ID NO: 33 - protein, 19 aa, Artificial Sequence
  PT/53 HCDR2
LIRNKANGYTTKYAASVKG

SEQ ID NO: 34 - protein, 6 aa, Artificial Sequence
  PT/53 HCDR3
AVWFAY

SEQ ID NO: 35 - protein, 9 aa, Artificial Sequence
  PT/53 LCDR3
LQLVEYPYT

SEQ ID NO: 36 - protein, 447 aa, Artificial Sequence
  PT/53 HC
EVKLMESGGGLVQPGASLRLSCAASGFTFTDYYMSWVRQPPGKAPEWLALIRNKANGYTTKYAASVKGRFTISRDNSQNILYLQMNTLRAEDSATYYCVKAVWFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 37 - protein, 219 aa, Artificial Sequence
  PT/53 LC
DIVITQDELSNPVTSGESVSISCRSSKSLLYKDGKTYLNWFLQRPGQSPQLLIYLMSTRASGVSDRFSGSGSGTDFTLEISRVKAEDVGVYYCLQLVEYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 38 - protein, 115 aa, Artificial Sequence
  PT/81 VH
QVQLQQPGAELVRPGASVILSCKASGYTFTNSWIHWVKQRPGRVLEWIGRIDPNSGGTRYNENFKSKATLTVDKPSSTAYMQLSSLTSEDSAVYFCSRGVLHDYWGQGTTLTVSS

SEQ ID NO: 39 - protein, 112 aa, Artificial Sequence
  PT/81 VL
DIVITQDELSNPVTSGESVSISCRSSKSLLYKDGKTYLNWFLQRPGQSPQLLIYLMSTRASGVSDRFSVSGSGTDFTLEISRVQAEDVGVYYCQQLVEYPYTFGGGTKLEIK

SEQ ID NO: 40 - protein, 10 aa, Artificial Sequence
  PT/81 HCDR1
GYTFTNSWIH

SEQ ID NO: 41 - protein, 17 aa, Artificial Sequence
  PT/81 HCDR2
RIDPNSGGTRYNENFKS

SEQ ID NO: 42 - protein, 6 aa, Artificial Sequence
  PT/81 HCDR3
GVLHDY

SEQ ID NO: 43 - protein, 9 aa, Artificial Sequence
  PT/81 LCDR3
QQLVEYPYT

SEQ ID NO: 44 - protein, 445 aa, Artificial Sequence
  PT/81 HC
QVQLQQPGAELVRPGASVILSCKASGYTFTNSWIHWVKQRPGRVLEWIGRIDPNSGGTRYNENFKSKATLTVDKPSSTAYMQLSSLTSEDSAVYFCSRGVLHDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 45 - protein, 219 aa, Artificial Sequence
  PT/81 LC
DIVITQDELSNPVTSGESVSISCRSSKSLLYKDGKTYLNWFLQRPGQSPQLLIYLMSTRASGVSDRFSVSGSGTDFTLEISRVQAEDVGVYYCQQLVEYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 46 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 47 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 48 - protein, 410 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 49 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 50 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 51 - protein, 441 aa, Homo sapiens; identical to SEQ ID NO: 50

SEQ ID NO: 52 - protein, 10 aa, Homo sapiens
EVSASLAKQG

SEQ ID NO: 53 - protein, 13 aa, Homo sapiens
ATLADEVSASLAK

SEQ ID NO: 54 - protein, 11 aa, Homo sapiens
ATLADEVSASL

SEQ ID NO: 55 - protein, 17 aa, Homo sapiens
SPQLATLADEVSASLAK

SEQ ID NO: 56 - protein, 9 aa, Homo sapiens
LADEVSASL

(19) United States
(12) Patent Application Publication
(10) Pub. No.: US 2024/0150451 A1
(43) Pub. Date: May 9, 2024

(54) ANTI-TAU ANTIBODIES AND USES THEREOF

(71) Applicant: Janssen Biotech, Inc., Horsham, PA (US)

(72) Inventors: Rupesh Nanjunda, Horsham, PA (US); Kristof Van Kolen, Horsham, PA (US)

(21) Appl. No.: 18/552,504

(22) PCT Filed: Mar. 25, 2022
(86) PCT No.: PCT/IB2022/052765

§ 371 Date: Sep. 26, 2023

Related U.S. Application Data
(60) Provisional application No. 63/196,365, filed on Jun. 3, 2021.
(60) Provisional application No. 63/166,439, filed on Mar. 26, 2021.

(51) Int. Cl. C07K 16/18; A61P 25/28; G01N 33/577

(52) U.S. Cl. CPC C07K 16/18; A61P 25/28; G01N 33/577; A61K 2039/505

(57) ABSTRACT

Monoclonal anti-PHF-tau antibodies and antigen-binding fragments thereof are described. Also described are nucleic acids encoding the antibodies, compositions comprising the antibodies, methods of producing the antibodies and using the antibodies for treating or preventing conditions such as tauopathies.

### FIELD OF THE INVENTION

The application relates to anti-PHF-tau antibodies, nucleic acids and expression vectors encoding the antibodies, recombinant cells containing the vectors, and compositions comprising the antibodies. Methods of making the antibodies, methods of using the antibodies to treat conditions including tauopathies, and methods of using the antibodies to diagnose diseases such as tauopathies are also provided.

### REFERENCE TO SEQUENCE LISTING SUBMITTED ELECTRONICALLY

This application contains a sequence listing, which is submitted electronically via EFS-Web as an ASCII formatted sequence listing with a file name “065768.96US2_Sequence_Listing” and a creation date of Jun. 3, 2021 and having a size of 66 kb. The sequence listing submitted via EFS-Web is part of the specification and is herein incorporated by reference in its entirety.

### BACKGROUND OF THE INVENTION

Alzheimer's Disease (AD) is a degenerative brain disorder characterized clinically by progressive loss of memory, cognition, reasoning, judgment and emotional stability that gradually leads to profound mental deterioration and ultimately death. AD is a very common cause of progressive mental failure (dementia) in aged humans and is believed to represent the fourth most common medical cause of death in the United States. AD has been observed in ethnic groups worldwide and presents a major present and future public health problem.

The brains of individuals with AD exhibit characteristic lesions termed senile (or amyloid) plaques, amyloid angiopathy (amyloid deposits in blood vessels) and neurofibrillary tangles. Large numbers of these lesions, particularly amyloid plaques and neurofibrillary tangles of paired helical filaments, are generally found in several areas of the human brain important for memory and cognitive function in patients with AD.

The current AD treatment landscape includes only therapies approved to treat cognitive symptoms in patients with dementia. There are no approved therapies that modify or slow the progression of AD. Potential disease modifiers are anti amyloid antibodies including Eli Lilly's Donanemab, a humanized IgG1 monoclonal antibody recognizing Aβ(p3-42), a pyroglutamate form of Aβ, and aducanumab, a human IgG1 monoclonal antibody against a conformational epitope found on Aβ. These therapies, and most other potential disease modifiers that may launch in the next decade, target A_{β} (the principle component of the amyloid plaques that are one of the two “hallmark” pathological signs of AD).

Neurofibrillary tangles, the second hallmark pathological sign of AD, are primarily composed of aggregates of hyper-phosphorylated tau protein. The main physiological function of tau is microtubule polymerization and stabilization. The binding of tau to microtubules takes place by ionic interactions between positive charges in the microtubule binding region of tau and negative charges on the microtubule lattice (Butner and Kirschner, J Cell Biol. 115(3):717-30, 1991). Tau protein contains 85 possible phosphorylation sites, and phosphorylation at many of these sites interferes with the primary function of tau. Tau that is bound to the axonal microtubule lattice is in a hypo-phosphorylation state, while aggregated tau in AD is hyper-phosphorylated, providing unique epitopes that are distinct from the physiologically active pool of tau.

A tauopathy transmission and spreading hypothesis has been described and is based on the Braak stages of tauopathy progression in the human brain and tauopathy spreading after tau aggregate injections in preclinical tau models (Frost et al., J Biol Chem. 284:12845-52, 2009; Clavaguera et al., Nat Cell Biol. 11:909-13, 2009).

Developing therapeutics preventing or clearing tau aggregation has been of interest for many years and candidate drugs, including anti-aggregation compounds and kinase inhibitors, have entered in clinical testing (Brunden et al., Nat Rev Drug Discov. 8:783-93, 2009). Multiple studies have been published that show the beneficial therapeutic effects of both active and passive tau immunization in transgenic mouse models (Chai et al., J Biol Chem. 286:34457-67, 2011; Boutajangout et al., J Neurochem. 118:658-67, 2011; Boutajangout et al., J Neurosci. 30:16559-66, 2010; Asuni et al., J Neurosci. 27:9115-29, 2007). Activity has been reported with both phospho-directed and non-phospho-directed antibodies (Schroeder et al., J Neuroimmune Pharmacol. 11(1):9-25, 2016).

Despite the progress, there remains a need for effective therapeutics that prevent tau aggregation and tauopathy progression to treat tauopathies such as AD, and other neurodegenerative diseases.

### BRIEF SUMMARY OF THE INVENTION

The application satisfies this need by providing anti-PHF-tau antibodies or antigen-binding fragments thereof that have high binding affinity towards paired helical filament (PHF)-tau and are selective for phosphorylated tau. Antibodies of the application were generated by human framework adaptation (HFA) of mouse PHF-tau-specific antibodies. It is thought that the selectivity of the antibodies for phosphorylated tau allows for efficacy against pathogenic tau without interfering with normal tau function. The application also provides nucleic acids encoding the antibodies, compositions comprising the antibodies, and methods of making and using the antibodies. Anti-PHF-tau antibodies or antigen-binding fragments thereof of the application inhibit tau seeds, as measured by cellular assays using tau seeds derived from HEK cell lysates or from spinal cord lysates from mutant tau transgenic mice. In addition, a chimeric antibody with variable regions of anti-PHF-tau antibodies of the application and mouse Ig constant regions, such as mouse IgG2a constant regions, blocked seeding activity in an in vivo mutant tau transgenic mouse model.

The progression of tauopathy in an AD brain follows distinct special spreading patterns. It has been shown in preclinical models that extracellular phospho-tau seeds can induce tauopathy in neurons (Clavaguera et al., PNAS 110(23):9535-40, 2013). It is therefore believed that tauopathy can spread in a prion-like fashion from one brain region to the next. This spreading process would involve an externalization of tau seeds that can be taken up by nearby neurons and induce further tauopathy. While not wishing to be bound by theory, it is thought that anti-PHF-tau antibodies or antigen-binding fragments thereof of the application prevent tau aggregation or the spreading of tauopathy in the brain by interacting with phospho-tau seeds.

In one general aspect, the application relates to an isolated monoclonal antibody or an antigen-binding fragment thereof that binds PHF-tau.

In another general aspect, the application relates to an isolated monoclonal antibody or antigen-binding fragment thereof that binds to a tau protein at an epitope of the tau protein consisting of or within the amino acid sequence of SEQ ID NO: 1, wherein the antibody or antigen-binding fragment thereof binds paired helical filament (PHF)-tau, preferably human PHF-tau.

According to a particular aspect, the application relates to an isolated monoclonal antibody or antigen-binding fragment thereof, wherein:
(a) the epitope of the tau protein comprises either one of phosphorylated S433 or phosphorylated S435 of the tau protein, but does not comprise phosphorylated S433 and phosphorylated S435;
(b) the epitope of the tau protein comprises one or more of phosphorylated T427, phosphorylated S433 and phosphorylated S435 of the tau protein, but does not comprise all of phosphorylated T427, phosphorylated S433 and phosphorylated S435;
(c) the epitope of the tau protein comprises one or more of phosphorylated T427 and phosphorylated S433 of the tau protein, but does not comprise phosphorylated S435, and does not comprise all of phosphorylated T427, phosphorylated S433 and phosphorylated S435; or
(d) the epitope of the tau protein comprises phosphorylated T427 of the tau protein, but does not comprise phosphorylated S433 or phosphorylated S435.

According to another particular aspect, the isolated monoclonal antibody or antigen-binding fragment thereof comprises:
(a) immunoglobulin heavy chain HCDR1, HCDR2 and HCDR3 having the polypeptide sequences of SEQ ID NOs: 4, 5 and 6, respectively; and immunoglobulin light chain LCDR1, LCDR2 and LCDR3 having the polypeptide sequences of SEQ ID NOs: 7, 8 and 9, respectively;
(b) immunoglobulin heavy chain HCDR1, HCDR2 and HCDR3 having the polypeptide sequences of SEQ ID NOs: 14, 15 and 16, respectively; and immunoglobulin light chain LCDR1, LCDR2 and LCDR3 having the polypeptide sequences of SEQ ID NOs: 17, 18 and 19, respectively;
(c) immunoglobulin heavy chain HCDR1, HCDR2 and HCDR3 having the polypeptide sequences of SEQ ID NOs: 24, 25 and 26, respectively; and immunoglobulin light chain LCDR1, LCDR2 and LCDR3 having the polypeptide sequences of SEQ ID NOs: 27, 18 and 19, respectively;
(d) immunoglobulin heavy chain HCDR1, HCDR2 and HCDR3 having the polypeptide sequences of SEQ ID NOs: 32, 33 and 34, respectively; and immunoglobulin light chain LCDR1, LCDR2 and LCDR3 having the polypeptide sequences of SEQ ID NOs: 17, 18 and 35, respectively; or
(e) immunoglobulin heavy chain HCDR1, HCDR2 and HCDR3 having the polypeptide sequences of SEQ ID NOs: 40, 41 and 42, respectively; and immunoglobulin light chain LCDR1, LCDR2 and LCDR3 having the polypeptide sequences of SEQ ID NOs: 17, 18 and 43, respectively.

According to another particular aspect, the isolated monoclonal antibody or antigen-binding fragment thereof comprises a heavy chain variable region having a polypeptide sequence at least 90% identical to SEQ ID NO: 2, 12, 22, 30 or 38, or a light chain variable region having a polypeptide sequence at least 90% identical to SEQ ID NO: 3, 13, 23, 31 or 39.

According to another particular aspect, the isolated monoclonal antibody or antigen-binding fragment thereof comprises a heavy chain variable region having a polypeptide sequence of SEQ ID NO: 2, 12, 22, 30 or 38, or a light chain variable region having a polypeptide sequence of SEQ ID NO: 3, 13, 23, 31 or 39.

According to another particular aspect, the isolated monoclonal antibody or antigen-binding fragment thereof comprises:
(a) a heavy chain variable region having the polypeptide sequence of SEQ ID NO: 2, and a light chain variable region having the polypeptide sequence of SEQ ID NO: 3;
(b) a heavy chain variable region having the polypeptide sequence of SEQ ID NO: 12, and a light chain variable region having the polypeptide sequence of SEQ ID NO: 13;
(c) a heavy chain variable region having the polypeptide sequence of SEQ ID NO: 22, and a light chain variable region having the polypeptide sequence of SEQ ID NO: 23;
(d) a heavy chain variable region having the polypeptide sequence of SEQ ID NO: 30, and a light chain variable region having the polypeptide sequence of SEQ ID NO: 31; or
(e) a heavy chain variable region having the polypeptide sequence of SEQ ID NO: 38, and a light chain variable region having the polypeptide sequence of SEQ ID NO: 39.

According to another particular aspect, the isolated monoclonal antibody or antigen-binding fragment thereof comprises:
(a) a heavy chain having the polypeptide sequence of SEQ ID NO: 10, and a light chain having the polypeptide sequence of SEQ ID NO: 11;
(b) a heavy chain having the polypeptide sequence of SEQ ID NO: 20, and a light chain having the polypeptide sequence of SEQ ID NO: 21;
(c) a heavy chain having the polypeptide sequence of SEQ ID NO: 28, and a light chain having the polypeptide sequence of SEQ ID NO: 29;
(d) a heavy chain having the polypeptide sequence of SEQ ID NO: 36, and a light chain having the polypeptide sequence of SEQ ID NO: 37; or
(e) a heavy chain having the polypeptide sequence of SEQ ID NO: 44, and a light chain having the polypeptide sequence of SEQ ID NO: 45.

In another general aspect, the application relates to an isolated monoclonal antibody or antigen-binding fragment thereof, comprising:
(a) an immunoglobulin heavy chain HCDR1, HCDR2 and HCDR3 having the polypeptide sequences of SEQ ID NOs: 4, 5 and 6, respectively; and immunoglobulin light chain LCDR1, LCDR2 and LCDR3 having the polypeptide sequences of SEQ ID NOs: 7, 8 and 9, respectively;
(b) a heavy chain variable region having the polypeptide sequence of SEQ ID NO: 2, and a light chain variable region having the polypeptide sequence of SEQ ID NO: 3; or
(c) a heavy chain having the polypeptide sequence of SEQ ID NO: 10, and a light chain having the polypeptide sequence of SEQ ID NO: 11.

In another general aspect, the application relates to an isolated monoclonal antibody or antigen-binding fragment thereof, comprising:
(a) immunoglobulin heavy chain HCDR1, HCDR2 and HCDR3 having the polypeptide sequences of SEQ ID NOs: 14, 15 and 16, respectively; and immunoglobulin light chain LCDR1, LCDR2 and LCDR3 having the polypeptide sequences of SEQ ID NOs: 17, 18 and 19, respectively;
(b) a heavy chain variable region having the polypeptide sequence of SEQ ID NO: 12, and a light chain variable region having the polypeptide sequence of SEQ ID NO: 13; or
(c) a heavy chain having the polypeptide sequence of SEQ ID NO: 20, and a light chain having the polypeptide sequence of SEQ ID NO: 21.

In another general aspect, the application relates to an isolated nucleic acid encoding the isolated monoclonal antibody or antigen-binding fragment thereof of the application.

In another general aspect, the application relates to a vector comprising an isolated nucleic acid encoding a monoclonal antibody or antigen-binding fragment thereof of the application.

In another general aspect, the application relates to a host cell comprising an isolated nucleic acid encoding a monoclonal antibody or antigen-binding fragment thereof of the application.

In another general aspect, the application relates to a pharmaceutical composition comprising an isolated monoclonal antibody or antigen-binding fragment thereof of the application and a pharmaceutically acceptable carrier.

In another general aspect, the application relates to a method of blocking tau seeding in a subject in need thereof, comprising administering to the subject a pharmaceutical composition of the application.

In another general aspect, the application relates to a method of treating a tauopathy in a subject in need thereof, comprising administering to the subject a pharmaceutical composition of the application. The tauopathy includes, but is not limited to, one or more selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

In another general aspect, the application relates to a method of reducing pathological tau aggregation or spreading of tauopathy in a subject in need thereof, comprising administering to the subject a pharmaceutical composition of the application. The tauopathy includes, but is not limited to, one or more selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

In another general aspect, the application relates to a method of producing a monoclonal antibody or antigen-binding fragment thereof of the application, comprising culturing a cell comprising a nucleic acid encoding the monoclonal antibody or antigen-binding fragment thereof under conditions to produce the monoclonal antibody or antigen-binding fragment thereof, and recovering the monoclonal antibody or antigen-binding fragment thereof from the cell or cell culture.

In another general aspect, the application relates to a method of detecting the presence of PHF-tau in a biological sample from a subject, comprising contacting the biological sample with a monoclonal antibody or antigen-binding fragment thereof of the application, and detecting binding of the monoclonal antibody or antigen-binding fragment thereof to PHF-tau in the sample from the subject. The biological sample includes, but is not limited to, one or more selected from the group consisting of a blood, serum, plasma, interstitial fluid, or cerebral spinal fluid sample.

Other aspects, features and advantages of the invention according to embodiments of the application will be apparent from the following disclosure, including the detailed description of the application and its preferred embodiments and the appended claims.

### BRIEF DESCRIPTION OF THE DRAWINGS

The foregoing summary, as well as the following detailed description of the application, will be better understood when read in conjunction with the appended drawings. It should be understood that the application is not limited to the precise embodiments shown in the drawings.

FIG. 1 shows binding of recombinantly expressed PT66, PT69/PT87, hTau60 to recombinant 2N4R tau analyzed by ELISA.

FIG. 2 shows western blot profiling of PT66, hTau60 and PT69/PT87 on brain extracts from (from left to right): WT (slot 1) and Tau −/− (slot 2) mouse brain, dog brain (slot 3), monkey brain (slot 4), and human brain (slot 5), and a PHF prep derived from postmortem AD brain (slot 6).

FIG. 3 shows representative SPR binding data of PT66, hTau60 and PT69/PT87 monoclonal antibodies (mAbs) to PHF-Tau and their respective Fab fragments to PHF-Tau and recombinant Tau. SPR binding sensorgrams of anti-tau antibodies and their corresponding Fab fragments against PHF-Tau and full-length rec. Tau protein. Individual traces within each sensorgram represent different concentrations of antibodies or Fabs injected. The individual traces correspond to 75 nM, 15 nM, 3 nM, 0.6 nM and 0.12 nM from top to bottom. For HT7, the top trace (concentration) is 15 nM. The solid black lines indicate global kinetics fitting with either bivalent analyte model (for mAbs with PHF-Tau) or 1:1 Langmuir model (for Fabs with PHF-Tau and recombinant Tau). For HT7, Fab fragment is not available.

FIG. 4 shows binding data on cryosections of on AD and non-AD brain of PT66, hTau60 and PT69/PT87.

FIG. 5 shows IHC profiling data from PT66, hTau60 and PT69/PT87. Binding on paraffin sections from WT, Tau −/− and P301S mice is presented.

FIG. 6A shows a schematic of the immunodepletion assay.

FIG. 6B shows the results of immunodepletion assay on the tested antibodies (hTau60, PT69, PT66 and an internal N-term binding tau mAb (PT26) tau seeds derived from the human AD brain tissue (square) and P301S spinal cord (triangle). PT66, hTau60 and PT69/PT87 inhibited tau seeding more effectively than the N-term antibody, as determined using the FRET assay. The immunodepleted fractions from human AD brain homogenates were also analyzed with a hTau60/hTau60 tau aggregate-specific MSD assay (circle).

FIG. 6C shows the results of a sequential immunodepletion (ID) assay, wherein the first round of immunodepletion assay (ID1) was conducted with each of the antibodies PT93 (targeting N-terminal portion of Tau), PT51 (a HT7-like antibody) and hTau60 (targeting C-terminal portion of Tau) or without any antibody (no mAb), and the second round of immunodepletion assay (ID2) was conducted with the same or different antibody as that used for ID1. It was shown that ID2 with same antibody used for ID1 did not deplete additional aggregates, and that after ID1 with PT93, ID2 with PT51 (HT7-like) and hTau60 (C-term) resulted in additional depletion of the Tau aggregates with hTau60 depleted all remaining aggregates.

FIG. 7A shows efficacy of hTau60 and PT69/PT87, compared to AT120, PT/76 and PT53 antibodies in an ePHF injection model (see, e.g., U.S. Pat. No. 10,766,953) co-injected with Tau PHFs and test antibodies. *** P<0.0001 Bonferroni multiple comparisons.

FIG. 7B shows efficacy of antibodies that bind to C-terminal PHF-tau (C-term) PT81 and PT66 compared to antibodies that bind to other epitopes in the N-terminal (N-term) or middle (Mid) portion of Tau, in the ePHF injection model. The C-terminal antibodies showed strong reduction in tau seeding in vivo.

FIG. 7C shows that both C-terminal antibodies (PT66 and hTau60) and PT3 retained in vivo activity after i.p. dosing. Mice were injected with 20 mg/kg antibody 2×/week.

FIG. 8 shows epitope mapping data using linear peptide mapping of internal C-terminal antibodies.

FIG. 9 shows that the lower efficacy by N-terminal antibodies can be explained by more extensive processing at N-terminus of PHF-tau: C-terminal PHF-tau antibodies PT66 and hTau60 (C-term) are compared to N-terminal PHF-tau antibodies (PT93) in a Western blotting screen analyzing PHF-tau.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the application. Such discussion is not an admission that any or all of these matters form part of the prior art with respect to any inventions disclosed or claimed.

### Definitions

Unless defined otherwise, all technical and scientific terms used herein have the same meaning commonly understood to one of ordinary skill in the art to which this application pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Unless otherwise stated, any numerical value, such as a concentration or a concentration range described herein, is to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the application can be replaced with the term “consisting of” or “consisting essentially of” to vary scopes of the disclosure.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids.

As used herein, the term “antibody” or “immunoglobulin” is used in a broad sense and includes immunoglobulin or antibody molecules including polyclonal antibodies, monoclonal antibodies including murine, human, human-adapted, humanized and chimeric monoclonal antibodies and antibody fragments.

In general, antibodies are proteins or peptide chains that exhibit binding specificity to a specific antigen. Antibody structures are well known. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibodies of the application include those that have variations in their Fc region such that they have altered properties as compared to wild type Fc regions including, but not limited to, extended half-life, reduced or increased ADCC or CDC and silenced Fc effector functions. Accordingly, the antibodies of the application can be of any of the five major classes or corresponding sub-classes. Preferably, the antibodies of the application are IgG1, IgG2, IgG3 or IgG4. Antibody light chains of any vertebrate species can be assigned to one of two clearly distinct types, namely kappa and lambda, based on the amino acid sequences of their constant domains. Accordingly, the antibodies of the application can contain a kappa or lambda light chain constant domain. According to particular embodiments, the antibodies of the application include heavy and/or light chain constant regions from mouse antibodies or human antibodies.

In addition to the heavy and light chain constant domains, antibodies contain light and heavy chain variable regions. An immunoglobulin light or heavy chain variable region consists of a “framework” region interrupted by “antigen-binding sites.” The antigen-binding sites are defined using various terms and numbering schemes as follows:
(i) Kabat: “Complementarity Determining Regions” or “CDRs” are based on sequence variability (Wu and Kabat, J Exp Med. 132:211-50, 1970). Generally, the antigen-binding site has three CDRs in each variable region (e.g., HCDR1, HCDR2 and HCDR3 in the heavy chain variable region (VH) and LCDR1, LCDR2 and LCDR3 in the light chain variable region (VL));
(ii) Chothia: The term “hypervariable region,” “HVR” or “HV” refers to the regions of an antibody variable domain which are hypervariable in structure as defined by Chothia and Lesk (Chothia and Lesk, J Mol Biol. 196:901-17, 1987). Generally, the antigen-binding site has three hypervariable regions in each VH (H1, H2, H3) and VL (L1, L2, L3). Numbering systems as well as annotation of CDRs and HVs have been revised by Abhinandan and Martin (Abhinandan and Martin, Mol Immunol. 45:3832-9, 2008);
(iii) IMGT: Another definition of the regions that form the antigen-binding site has been proposed by Lefranc (Lefranc et al., Dev Comp Immunol. 27:55-77, 2003) based on the comparison of V domains from immunoglobulins and T-cell receptors. The International ImMunoGeneTics (IMGT) database provides a standardized numbering and definition of these regions. The correspondence between CDRs, HVs and IMGT delineations is described in Lefranc et al., 2003, Id.;
(iv) AbM: A compromise between Kabat and Chothia numbering schemes is the AbM numbering convention described by Martin (Martin ACR (2010) Antibody Engineering, eds Kontermann R, Dubel S (Springer-Verlag, Berlin), Vol 2, pp 33-51);
(v) The antigen-binding site can also be delineated based on “Specificity Determining Residue Usage” (SDRU) (Almagro, Mol Recognit. 17:132-43, 2004), where SDR, refers to amino acid residues of an immunoglobulin that are directly involved in antigen contact.

“Framework” or “framework sequence” is the remaining sequences within the variable region of an antibody other than those defined to be antigen-binding site sequences. Because the exact definition of an antigen-binding site can be determined by various delineations as described above, the exact framework sequence depends on the definition of the antigen-binding site. The framework regions (FRs) are the more highly conserved portions of variable domains. The variable domains of native heavy and light chains each comprise four FRs (FR1, FR2, FR3 and FR4, respectively) which generally adopt a beta-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227: 799-817, 1992; Tramontano et al., J. Mol. Biol. 215:175-182, 1990). Despite their high sequence variability, five of the six loops adopt just a small repertoire of main-chain conformations, called “canonical structures.” These conformations are first of all determined by the length of the loops and secondly by the presence of key residues at certain positions in the loops and in the framework regions that determine the conformation through their packing, hydrogen bonding or the ability to assume unusual main-chain conformations.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the constant region of the heavy chain. According to other particular embodiments, the antigen-binding fragment comprises Fab and F(ab′).

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “epitope” refers to a site on an antigen to which an immunoglobulin, antibody, or antigen-binding fragment thereof, specifically binds. Epitopes can be formed either from contiguous amino acids or from noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

As used herein, the term “tau” or “tau protein” refers to an abundant central and peripheral nervous system protein having multiple isoforms. In the human central nervous system (CNS), six major tau isoforms ranging in size from 352 to 441 amino acids in length exist due to alternative splicing (Hanger et al., Trends Mol Med. 15:112-9, 2009). The isoforms differ from each other by the regulated inclusion of 0-2 N-terminal inserts, and 3 or 4 tandemly arranged microtubule-binding repeats, and are referred to as 0N3R (SEQ ID NO: 46), 1N3R (SEQ ID NO: 47), 2N3R (SEQ ID NO: 48), 0N4R (SEQ ID NO: 49), 1N4R (SEQ ID NO: 50) and 2N4R (SEQ ID NO: 51). As used herein, the term “control tau” refers to the tau isoform of SEQ ID NO: 51 that is devoid of phosphorylation and other post-translational modifications. As used herein, the term “tau” includes proteins comprising mutations, e.g., point mutations, fragments, insertions, deletions and splice variants of full-length wild type tau. The term “tau” also encompasses post-translational modifications of the tau amino acid sequence. Post-translational modifications include, but are not limited to, phosphorylation. As used herein, the phrase “phosphorylated S433 of the tau protein” and similar phrases refer to a phosphorylated amino acid at a certain position, e.g., serine at position 433, of the full-length wild type tau protein.

Tau binds microtubules and regulates transport of cargo through cells, a process that can be modulated by tau phosphorylation. In AD and related disorders, abnormal phosphorylation of tau is prevalent and thought to precede and/or trigger aggregation of tau into fibrils, termed paired helical filaments (PHF). The major constituent of PHF is hyper-phosphorylated tau. As used herein, the term “paired helical filament-tau” or “PHF-tau” refers to tau aggregates in paired helical filaments. Two major regions in PHF structure are evident in electron microscopy, the fuzzy coat and the core filament; the fuzzy coat being sensitive to proteolysis and located outside of the filaments, and the protease-resistant core of filaments forming the backbone of PHFs (Wischik et al. Proc Natl Acad Sci USA. 85:4884-8, 1988).

An “isolated monoclonal antibody that binds PHF-tau” or an “isolated monoclonal anti-PHF-tau antibody,” as used herein, is intended to refer to a monoclonal anti-PHF-tau antibody which is substantially free of other antibodies having different antigenic specificities (for instance, an isolated monoclonal anti-PHF-tau antibody is substantially free of antibodies that specifically bind antigens other than PHF-tau). An isolated monoclonal anti-PHF-tau antibody can, however, have cross-reactivity to other related antigens, for instance from other species (such as PHF-tau species homologs).

As used herein, the term “specifically binds” or “specific binding” refers to the ability of an anti-PHF-tau antibody of the application to bind to a predetermined target with a dissociation constant (K_{D}) of about 1×10⁻⁶ M or tighter, for example, about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. The KD is obtained from the ratio of Kd to Ka (i.e., Kd/Ka) and is expressed as a molar concentration (M). KD values for antibodies can be determined using methods in the art in view of the present disclosure. For example, the KD value of an anti-PHF-tau antibody can be determined by using surface plasmon resonance, such as by using a biosensor system, e.g., a Biacore® system, a ProteOn instrument (BioRad), a KinExA instrument (Sapidyne), ELISA or competitive binding assays known to those skilled in the art. Typically, an anti-PHF-tau antibody binds to a predetermined target (i.e. PHF-tau) with a K_{D} that is at least ten fold less than its K_{D} for a nonspecific target as measured by surface plasmon resonance using, for example, a ProteOn Instrument (BioRad). The anti-PHF-tau antibodies that specifically bind to PHF-tau can, however, have cross-reactivity to other related targets, for example, to the same predetermined target from other species (homologs).

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the application. The “host cell” can be any type of cell, e.g., a primary cell, a cell in culture, or a cell from a cell line. In one embodiment, a “host cell” is a cell transfected with a nucleic acid molecule of the application. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed monoclonal antibody or antigen-binding fragment thereof that binds PHF-tau can be within the cytoplasm of a host cell, into the extracellular milieu such as the growth medium of a cell culture, or anchored to the cell membrane.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the application or the biological activity of a composition according to the application. According to particular embodiments, in view of the present disclosure, any pharmaceutically acceptable carrier suitable for use in an antibody pharmaceutical composition can be used in the invention.

As used herein, the term “subject” refers to an animal, and preferably a mammal. According to particular embodiments, the subject is a mammal including a non-primate (e.g., a camel, donkey, zebra, cow, pig, horse, goat, sheep, cat, dog, rat, rabbit, guinea pig or mouse) or a primate (e.g., a monkey, chimpanzee, or human). In particular embodiments, the subject is a human.

As used herein, the term “therapeutically effective amount” refers to an amount of an active ingredient or component that elicits the desired biological or medicinal response in a subject. A therapeutically effective amount can be determined empirically and in a routine manner, in relation to the stated purpose. For example, in vitro assays can optionally be employed to help identify optimal dosage ranges. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the route of administration, and the severity of disease, and should be decided according to the judgment of the practitioner and each patient's circumstances. Effective doses can be extrapolated from dose-response curves derived from in vitro or animal model test systems.

As used herein, the terms “treat,” “treating,” and “treatment” are all intended to refer to an amelioration or reversal of at least one measurable physical parameter related to a tauopathy which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the tauopathy. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

As used herein a “tauopathy” encompasses any neurodegenerative disease that involves the pathological aggregation of tau within the brain. In addition to familial and sporadic AD, other exemplary tauopathies are frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, and chronic traumatic encephalopathy, such as dementia pugulistica (boxing disease) (Morris et al., Neuron, 70:410-26, 2011).

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### Anti-PHF-Tau Antibodies

In one general aspect, the application relates to isolated monoclonal antibodies or antigen-binding fragments thereof that bind PHF-tau. Such anti-PHF-tau antibodies can have the properties of binding a phosphorylated epitope on PHF-tau or binding to a non-phosphorylated epitope on PHF-tau. Anti-PHF-tau antibodies can be useful as therapeutics, and as research or diagnostic reagents to detect PHF-tau in biological samples, for example in tissues or cells.

According to a particular aspect, the application relates to an isolated antibody or an antigen-binding fragment thereof that binds to a tau protein at an epitope in the C-terminus domain of the tau protein. In some embodiments, the isolated monoclonal antibody or antigen-binding fragment thereof binds to a tau protein at an epitope of the tau protein having or within the amino acid sequence of SEQ ID NO: 1, wherein the antibody or antigen-binding fragment thereof binds PHF-tau, preferably human PHF-tau. Preferably, the isolated monoclonal antibody or antigen-binding fragment thereof binds to a tau protein at an epitope of the tau protein consisting of or within the amino acid sequence of SEQ ID NO: 1, wherein the antibody or antigen-binding fragment thereof binds PHF-tau, preferably human PHF-tau.

In some embodiments, the epitope of the tau protein comprises either one of phosphorylated S433 or phosphorylated S435 of the tau protein, but does not comprise phosphorylated S433 and phosphorylated S435; or the epitope of the tau protein comprises one or more of phosphorylated T427, phosphorylated S433 and phosphorylated S435 of the tau protein, but does not comprise all of phosphorylated T427, phosphorylated S433 and phosphorylated S435; or the epitope of the tau protein comprises one or more of phosphorylated T427 and phosphorylated S433 of the tau protein, but does not comprise phosphorylated S435, and does not comprise all of phosphorylated T427, phosphorylated S433 and phosphorylated S435; or the epitope of the tau protein comprises phosphorylated T427 of the tau protein, but does not comprise phosphorylated S433 or phosphorylated S435.

Antibodies of the present invention can be produced by a variety of techniques, for example by the hybridoma method (Kohler and Milstein, Nature. 256:495-7, 1975). Chimeric monoclonal antibodies containing a light chain and heavy chain variable region derived from a donor antibody (typically murine) in association with light and heavy chain constant regions derived from an acceptor antibody (typically another mammalian species such as human) can be prepared by a method disclosed in U.S. Pat. No. 4,816,567. CDR-grafted monoclonal antibodies having CDRs derived from a non-human donor immunoglobulin (typically murine) and the remaining immunoglobulin-derived parts of the molecule being derived from one or more human immunoglobulins can be prepared by techniques known to those skilled in the art such as that disclosed in U.S. Pat. No. 5,225,539. Fully human monoclonal antibodies lacking any non-human sequences can be prepared from human immunoglobulin transgenic mice by techniques referenced in Lonberg et al., Nature. 368:856-9, 1994; Fishwild et al., Nat Biotechnol. 14:845-51, 1996; and Mendez et al., Nat Genet. 15:146-56, 1997. Human monoclonal antibodies can also be prepared and optimized from phage display libraries (see, e.g., Knappik et al., J Mol Biol. 296:57-86, 2000; Krebs et al., J Immunol Methods. 254:67-84, 2001; Shi et al., J Mol Biol. 397:385-96, 2010).

According to a particular aspect, the application relates to isolated monoclonal antibodies or antigen-binding fragments thereof comprising:
(a) immunoglobulin heavy chain HCDR1, HCDR2 and HCDR3 having the polypeptide sequences of SEQ ID NOs: 4, 5 and 6, respectively; and immunoglobulin light chain LCDR1, LCDR2 and LCDR3 having the polypeptide sequences of SEQ ID NOs: 7, 8 and 9, respectively;
(b) immunoglobulin heavy chain HCDR1, HCDR2 and HCDR3 having the polypeptide sequences of SEQ ID NOs: 14, 15 and 16, respectively; and immunoglobulin light chain LCDR1, LCDR2 and LCDR3 having the polypeptide sequences of SEQ ID NOs: 17, 18 and 19, respectively;
(c) immunoglobulin heavy chain HCDR1, HCDR2 and HCDR3 having the polypeptide sequences of SEQ ID NOs: 24, 25 and 26, respectively; and immunoglobulin light chain LCDR1, LCDR2 and LCDR3 having the polypeptide sequences of SEQ ID NOs: 27, 18 and 19, respectively;
(d) immunoglobulin heavy chain HCDR1, HCDR2 and HCDR3 having the polypeptide sequences of SEQ ID NOs: 32, 33 and 34, respectively; and immunoglobulin light chain LCDR1, LCDR2 and LCDR3 having the polypeptide sequences of SEQ ID NOs: 17, 18 and 35, respectively; or
(e) immunoglobulin heavy chain HCDR1, HCDR2 and HCDR3 having the polypeptide sequences of SEQ ID NOs: 40, 41 and 42, respectively; and immunoglobulin light chain LCDR1, LCDR2 and LCDR3 having the polypeptide sequences of SEQ ID NOs: 17, 18 and 43, respectively.

Provided herein are isolated monoclonal antibodies or antigen-binding fragments thereof comprising a heavy chain variable region having a polypeptide sequence at least 90% identical to SEQ ID NO: 2, 12, 22, 30 or 38, or a light chain variable region having a polypeptide sequence at least 90% identical to SEQ ID NO: 3, 13, 23, 31 or 39. In some embodiments, the isolated monoclonal antibody or antigen-binding fragment thereof comprises a heavy chain variable region having a polypeptide sequence of SEQ ID NO: 2, 12, 22, 30 or 38, or a light chain variable region having a polypeptide sequence of SEQ ID NO: 3, 13, 23, 31 or 39. In some embodiments, the isolated monoclonal antibody or antigen-binding fragment thereof comprises a heavy chain variable region consisting of a polypeptide sequence of SEQ ID NO: 2, 12, 22, 30 or 38, or a light chain variable region consisting of a polypeptide sequence of SEQ ID NO: 3, 13, 23, 31 or 39.

According to a particular aspect, the application relates to isolated monoclonal antibodies or antigen-binding fragments thereof comprising:
(a) a heavy chain variable region having a polypeptide sequence at least 90%, such as at least 90%, 91%, 92%, 93%, 94%, 95%, 96%, 97%, 98%, 99% or 100% identical to the amino acid sequence of SEQ ID NO: 2, and a light chain variable region having a polypeptide sequence at least 90%, such as at least 90%, 91%, 92%, 93%, 94%, 95%, 96%, 97%, 98%, 99% or 100% identical to the amino acid sequence of SEQ ID NO: 3;
(b) a heavy chain variable region having a polypeptide sequence at least 90%, such as at least 90%, 91%, 92%, 93%, 94%, 95%, 96%, 97%, 98%, 99% or 100% identical to the amino acid sequence of SEQ ID NO: 12, and a light chain variable region having a polypeptide sequence at least 90%, such as at least 90%, 91%, 92%, 93%, 94%, 95%, 96%, 97%, 98%, 99% or 100% identical to the amino acid sequence of SEQ ID NO: 13;
(c) a heavy chain variable region having a polypeptide sequence at least 90%, such as at least 90%, 91%, 92%, 93%, 94%, 95%, 96%, 97%, 98%, 99% or 100% identical to the amino acid sequence of SEQ ID NO: 22, and a light chain variable region having a polypeptide sequence at least 90%, such as at least 90%, 91%, 92%, 93%, 94%, 95%, 96%, 97%, 98%, 99% or 100% identical to the amino acid sequence of SEQ ID NO: 23;
(d) a heavy chain variable region having a polypeptide sequence at least 90%, such as at least 90%, 91%, 92%, 93%, 94%, 95%, 96%, 97%, 98%, 99% or 100% identical to the amino acid sequence of SEQ ID NO: 30, and a light chain variable region having a polypeptide sequence at least 90%, such as at least 90%, 91%, 92%, 93%, 94%, 95%, 96%, 97%, 98%, 99% or 100% identical to the amino acid sequence of SEQ ID NO: 31; or
(e) a heavy chain variable region having a polypeptide sequence at least 90%, such as at least 90%, 91%, 92%, 93%, 94%, 95%, 96%, 97%, 98%, 99% or 100% identical to the amino acid sequence of SEQ ID NO: 38, and a light chain variable region having a polypeptide sequence at least 90%, such as at least 90%, 91%, 92%, 93%, 94%, 95%, 96%, 97%, 98%, 99% or 100% identical to the amino acid sequence of SEQ ID NO: 39.

According to another particular aspect, the application relates to isolated monoclonal antibodies or antigen-binding fragments thereof comprising:
(a) a heavy chain variable region having, preferably consisting of, the polypeptide sequence of SEQ ID NO: 2, and a light chain variable region having the polypeptide sequence of SEQ ID NO: 3;
(b) a heavy chain variable region having, preferably consisting of, the polypeptide sequence of SEQ ID NO: 12, and a light chain variable region having, preferably consisting of, the polypeptide sequence of SEQ ID NO: 13;
(c) a heavy chain variable region having, preferably consisting of, the polypeptide sequence of SEQ ID NO: 22, and a light chain variable region having, preferably consisting of, the polypeptide sequence of SEQ ID NO: 23;
(d) a heavy chain variable region having, preferably consisting of, the polypeptide sequence of SEQ ID NO: 30, and a light chain variable region having, preferably consisting of, the polypeptide sequence of SEQ ID NO: 31; or
(e) a heavy chain variable region having, preferably consisting of, the polypeptide sequence of SEQ ID NO: 38, and a light chain variable region having, preferably consisting of, the polypeptide sequence of SEQ ID NO: 39.

According to another particular aspect, the application relates to isolated monoclonal antibodies or antigen-binding fragments thereof comprising:
(a) a heavy chain having a polypeptide sequence at least 90%, such as at least 90%, 91%, 92%, 93%, 94%, 95%, 96%, 97%, 98%, 99% or 100% identical to the amino acid sequence of SEQ ID NO: 10, and a light chain having a polypeptide sequence at least 90%, such as at least 90%, 91%, 92%, 93%, 94%, 95%, 96%, 97%, 98%, 99% or 100% identical to the amino acid sequence of SEQ ID NO: 11;
(b) a heavy chain having a polypeptide sequence at least 90%, such as at least 90%, 91%, 92%, 93%, 94%, 95%, 96%, 97%, 98%, 99% or 100% identical to the amino acid sequence of SEQ ID NO: 20, and a light chain having a polypeptide sequence at least 90%, such as at least 90%, 91%, 92%, 93%, 94%, 95%, 96%, 97%, 98%, 99% or 100% identical to the amino acid sequence of SEQ ID NO: 21;
(c) a heavy chain having a polypeptide sequence at least 90%, such as at least 90%, 91%, 92%, 93%, 94%, 95%, 96%, 97%, 98%, 99% or 100% identical to the amino acid sequence of SEQ ID NO: 28, and a light chain having a polypeptide sequence at least 90%, such as at least 90%, 91%, 92%, 93%, 94%, 95%, 96%, 97%, 98%, 99% or 100% identical to the amino acid sequence of SEQ ID NO: 29;
(d) a heavy chain having a polypeptide sequence at least 90%, such as at least 90%, 91%, 92%, 93%, 94%, 95%, 96%, 97%, 98%, 99% or 100% identical to the amino acid sequence of SEQ ID NO: 36, and a light chain having a polypeptide sequence at least 90%, such as at least 90%, 91%, 92%, 93%, 94%, 95%, 96%, 97%, 98%, 99% or 100% identical to the amino acid sequence of SEQ ID NO: 37; or
(e) a heavy chain having a polypeptide sequence at least 90%, such as at least 90%, 91%, 92%, 93%, 94%, 95%, 96%, 97%, 98%, 99% or 100% identical to the amino acid sequence of SEQ ID NO: 44, and a light chain having a polypeptide sequence at least 90%, such as at least 90%, 91%, 92%, 93%, 94%, 95%, 96%, 97%, 98%, 99% or 100% identical to the amino acid sequence of SEQ ID NO: 45.

According to another particular aspect, the application relates to isolated monoclonal antibodies or antigen-binding fragments thereof comprising:
(a) a heavy chain having, preferably consisting of, the polypeptide sequence of SEQ ID NO: 10, and a light chain having the polypeptide sequence of SEQ ID NO: 11;
(b) a heavy chain having, preferably consisting of, the polypeptide sequence of SEQ ID NO: 20, and a light chain having the polypeptide sequence of SEQ ID NO: 21;
(c) a heavy chain having, preferably consisting of, the polypeptide sequence of SEQ ID NO: 28, and a light chain having the polypeptide sequence of SEQ ID NO: 29;
(d) a heavy chain having, preferably consisting of, the polypeptide sequence of SEQ ID NO: 36, and a light chain having the polypeptide sequence of SEQ ID NO: 37; or
(e) a heavy chain having, preferably consisting of, the polypeptide sequence of SEQ ID NO: 44, and a light chain having the polypeptide sequence of SEQ ID NO: 45.

In some embodiments, an isolated monoclonal antibody or antigen-binding fragment thereof of the application comprises:
(a) an immunoglobulin heavy chain HCDR1, HCDR2 and HCDR3 having, preferably consisting of, the polypeptide sequences of SEQ ID NOs: 4, 5 and 6, respectively; and immunoglobulin light chain LCDR1, LCDR2 and LCDR3 having, preferably consisting of, the polypeptide sequences of SEQ ID NOs: 7, 8 and 9, respectively;
(b) a heavy chain variable region having, preferably consisting of, the polypeptide sequence of SEQ ID NO: 2, and a light chain variable region having, preferably consisting of, the polypeptide sequence of SEQ ID NO: 3; or
(c) a heavy chain having, preferably consisting of, the polypeptide sequence of SEQ ID NO: 10, and a light chain having, preferably consisting of, the polypeptide sequence of SEQ ID NO: 11.

In some embodiments, an isolated monoclonal antibody or antigen-binding fragment thereof of the application comprises:
(a) an immunoglobulin heavy chain HCDR1, HCDR2 and HCDR3 having, preferably consisting of, the polypeptide sequences of SEQ ID NOs: 14, 15 and 16, respectively; and immunoglobulin light chain LCDR1, LCDR2 and LCDR3 having, preferably consisting of, the polypeptide sequences of SEQ ID NOs: 17, 18 and 19, respectively;
(b) a heavy chain variable region having, preferably consisting of, the polypeptide sequence of SEQ ID NO: 12, and a light chain variable region having, preferably consisting of, the polypeptide sequence of SEQ ID NO: 13; or
(c) a heavy chain having the polypeptide sequence of SEQ ID NO: 20, and a light chain having the polypeptide sequence of SEQ ID NO: 21.

According to another particular aspect, the application relates to an isolated monoclonal antibody or antigen-binding fragment thereof, wherein the antibody or antigen-binding fragment binds to PHF-tau with a dissociation constant (KD) of 5×10-9 M or less, preferably a KD of 1×10-9 M or less or 1×10-10 M or less, wherein the KD is measured by surface plasmon resonance analysis, such as by using a Biacore or ProteOn system.

The functional activity of monoclonal antibodies and antigen-binding fragments thereof that bind PHF-tau can be characterized by methods known in the art and as described herein. Methods for characterizing antibodies and antigen-binding fragments thereof that bind PHF-tau include, but are not limited to, affinity and specificity assays including Biacore, ELISA, and FACS analysis; immunohistochemistry analysis; in vitro cellular assays and in vivo injection assays to determine the efficacy of the antibodies in inhibiting tau seeding; cell cytotoxicity assays to detect the presence of antibody-dependent cell-mediated cytotoxicity (ADCC), and complement dependent cytotoxicity (CDC) activity of the antibodies; etc. According to particular embodiments, methods for characterizing antibodies and antigen-binding fragments thereof that bind PHF-tau include those described in the Examples below. An exemplary mouse parental antibody of monoclonal antibodies binding PHF-tau but not control tau is antibody PT3, which is described in U.S. Pat. No. 9,371,376, the content of which is incorporated herein by reference in its entirety.

Several well-known methodologies can be employed to determine the binding epitope of the antibodies of the application. For example, when the structures of both individual components are known, in silico protein-protein docking can be carried out to identify compatible sites of interaction. Hydrogen-deuterium (H/D) exchange can be carried out with the antigen and antibody complex to map regions on the antigen that are bound by the antibody. Segment and point mutagenesis of the antigen can be used to locate amino acids important for antibody binding. The co-crystal structure of an antibody-antigen complex is used to identify residues contributing to the epitope and paratope. According to particular embodiments, methods for determining the binding epitope of antibodies of the application include those described in Examples below.

Antibodies of the application can be bispecific or multispecific. An exemplary bispecific antibody can bind two distinct epitopes on PHF-tau or can bind PHF-tau and amyloid beta (Abeta). Another exemplary bispecific antibody can bind PHF-tau and an endogenous blood-brain barrier transcytosis receptor such as insulin receptor, transferring receptor, insulin-like growth factor-1 receptor, and lipoprotein receptor. An exemplary antibody is of IgG1 type.

Immune effector properties of the antibodies of the application can be enhanced or silenced through Fc modifications by techniques known to those skilled in the art. For example, Fc effector functions such as Cl q binding, complement dependent cytotoxicity (CDC), antibody-dependent cell-mediated cytotoxicity (ADCC), phagocytosis, down regulation of cell surface receptors (e.g., B cell receptor; BCR), etc. can be provided and/or controlled by modifying residues in the Fc responsible for these activities. Pharmacokinetic properties can also be enhanced by mutating residues in the Fc domain that extend antibody half-life (Strohl, Curr Opin Biotechnol. 20:685-91, 2009).

Additionally, antibodies of the application can be post-translationally modified by processes such as glycosylation, isomerization, deglycosylation or non-naturally occurring covalent modification such as the addition of polyethylene glycol moieties and lipidation. Such modifications can occur in vivo or in vitro. For example, the antibodies of the application can be conjugated to polyethylene glycol (PEGylated) to improve their pharmacokinetic profiles. Conjugation can be carried out by techniques known to those skilled in the art. Conjugation of therapeutic antibodies with PEG has been shown to enhance pharmacodynamics while not interfering with function (Knight et al., Platelets. 15:409-18, 2004; Leong et al., Cytokine. 16:106-19, 2001; Yang et al., Protein Eng. 16:761-70, 2003).

In another general aspect, the application relates to an isolated polynucleotide encoding a monoclonal antibody or antigen-binding fragment thereof of the application. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding monoclonal antibodies or antigen-binding fragments thereof of the application can be altered without changing the amino acid sequences of the proteins. Exemplary isolated polynucleotides are polynucleotides encoding polypeptides comprising the immunoglobulin heavy chain and light chains described in the Examples (e.g., SEQ ID NOs: 10, 11, 20, 21, 28, 29, 36, 37, 44, 45) and polynucleotides encoding polypeptides comprising the heavy chain variable regions (VH) and light chain variable regions (VL) (e.g., SEQ ID NOs: 2, 3, 12, 13, 22, 23, 30, 31, 38, 39). Other polynucleotides which, given the degeneracy of the genetic code or codon preferences in a given expression system, encode the antibodies of the application are also within the scope of the application. The isolated nucleic acids of the present invention can be made using well known recombinant or synthetic techniques. DNA encoding the monoclonal antibodies is readily isolated and sequenced using methods known in the art. Where a hybridoma is produced, such cells can serve as a source of such DNA. Alternatively, display techniques wherein the coding sequence and the translation product are linked, such as phage or ribosomal display libraries, can be used.

In another general aspect, the application relates to a vector comprising an isolated polynucleotide encoding a monoclonal antibody or antigen-binding fragment thereof of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the application.

In another general aspect, the application relates to a host cell comprising an isolated polynucleotide encoding a monoclonal antibody or antigen-binding fragment thereof of the application. Any host cell known to those skilled in the art in view of the present disclosure can be used for recombinant expression of antibodies or antigen-binding fragments thereof of the application. Such host cells can be eukaryotic cells, bacterial cells, plant cells or archaeal cells. Exemplary eukaryotic cells can be of mammalian, insect, avian or other animal origins. Mammalian eukaryotic cells include immortalized cell lines such as hybridomas or myeloma cell lines such as SP2/0 (American Type Culture Collection (ATCC), Manassas, Va., CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1 SV (Lonza Biologics), CHO-K1 (ATCC CRL-61, Invitrogen) or DG44.

In another general aspect, the application relates to a method of producing a monoclonal antibody or antigen-binding fragment thereof of the application, comprising culturing a cell comprising a polynucleotide encoding the monoclonal antibody or antigen-binding fragment thereof under conditions to produce a monoclonal antibody or antigen-binding fragment thereof of the application, and recovering the antibody or antigen-binding fragment thereof from the cell or cell culture (e.g., from the supernatant). Expressed antibodies or antigen-binding fragments thereof can be harvested from the cells and purified according to conventional techniques known in the art.

### Pharmaceutical Compositions and Methods of Treatment

Anti-PHF-tau antibodies of the application or fragments thereof of the application can be used to treat, reduce or prevent symptoms in patients having a neurodegenerative disease that involves pathological aggregation of tau within the brain, or a tauopathy, such as patients suffering from AD.

Thus, in another general aspect, the application relates to a pharmaceutical composition comprising an isolated monoclonal antibody or antigen-binding fragment thereof of the application and a pharmaceutically acceptable carrier.

In another general aspect, the application relates to a method of blocking tau seeding in a subject in need thereof, comprising administering to the subject a pharmaceutical composition of the application. A “tau seed” as used herein refers to a tau aggregate capable of nucleating or “seeding” intracellular tau aggregation when internalized by a cell, or when exposed to monomeric tau in vitro. Tau seeding activity may be assessed in cellular tau aggregation assays as described herein (see also e.g., U.S. Pat. No. 9,834,596 which is incorporated by reference in its entirety).

In another general aspect, the application relates to a method of treating or reducing symptoms of a disease, disorder or condition, such as a tauopathy, in a subject in need thereof, comprising administering to the subject a pharmaceutical composition of the application.

In another general aspect, the application relates to a method of reducing pathological tau aggregation or spreading of tauopathy in a subject in need thereof, comprising administering to the subject a pharmaceutical composition of the application.

According to embodiments of the application, the pharmaceutical composition comprises a therapeutically effective amount of the monoclonal anti-PHF-tau antibody or antigen-binding fragment thereof. As used herein with reference to monoclonal anti-PHF-tau antibodies or antigen-binding fragments thereof, a therapeutically effective amount means an amount of the monoclonal anti-PHF-tau antibody or antigen-binding fragment thereof that results in treatment of a disease, disorder, or condition; prevents or slows the progression of the disease, disorder, or condition; or reduces or completely alleviates symptoms associated with the immune disease, disorder, or condition.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

According to particular embodiments, the disease, disorder or condition to be treated is a tauopathy. According to more particular embodiments, the disease, disorder or condition to be treated, includes, but is not limited to, familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, or dementia pugulistica (boxing disease).

A tauopathy-related behavioral phenotype includes, but is not limited to, cognitive impairments, early personality change and disinhibition, apathy, abulia, mutism, apraxia, perseveration, stereotyped movements/behaviors, hyperorality, disorganization, inability to plan or organize sequential tasks, selfishness/callousness, antisocial traits, a lack of empathy, halting, agrammatic speech with frequent paraphasic errors but relatively preserved comprehension, impaired comprehension and word-finding deficits, slowly progressive gait instability, retropulsions, freezing, frequent falls, non-levodopa responsive axial rigidity, supranuclear gaze palsy, square wave jerks, slow vertical saccades, pseudobulbar palsy, limb apraxia, dystonia, cortical sensory loss, and tremor.

Patients amenable to treatment include, but are not limited to, asymptomatic individuals at risk of AD or other tauopathy, as well as patients presently showing symptoms. Patients amenable to treatment include individuals who have a known genetic risk of AD, such as a family history of AD or presence of genetic risk factors in the genome. Exemplary risk factors are mutations in the amyloid precursor protein (APP), especially at position 717 and positions 670 and 671 (Hardy and Swedish mutations, respectively). Other risk factors are mutations in the presenilin genes PS1 and PS2 and in ApoE4, family history of hypercholesterolemia or atherosclerosis. Individuals presently suffering from AD can be recognized from characteristic dementia by the presence of risk factors described above. In addition, a number of diagnostic tests are available to identify individuals who have AD. These include measurement of cerebrospinal fluid tau and Abeta 42 levels. Elevated tau and decreased Abeta 42 levels signify the presence of AD. Individuals suffering from AD can also be diagnosed by AD and Related Disorders Association criteria.

Anti-PHF-tau antibodies of the application are suitable both as therapeutic and prophylactic agents for treating or preventing neurodegenerative diseases that involve pathological aggregation of tau, such as AD or other tauopathies. In asymptomatic patients, treatment can begin at any age (e.g., at about 10, 15, 20, 25, 30 years). Usually, however, it is not necessary to begin treatment until a patient reaches about 40, 50, 60, or 70 years. Treatment typically entails multiple dosages over a period of time. Treatment can be monitored by assaying antibody or activated T-cell or B-cell responses to the therapeutic agent over time. If the response falls, a booster dosage can be indicated.

In prophylactic applications, pharmaceutical compositions or medicaments are administered to a patient susceptible to, or otherwise at risk of, AD in an amount sufficient to eliminate or reduce the risk, lessen the severity, or delay the outset of the disease, including biochemical, histologic and/or behavioral symptoms of the disease, its complications and intermediate pathological phenotypes presented during development of the disease. In therapeutic applications, compositions or medicaments are administered to a patient suspected of, or already suffering from, such a disease in an amount sufficient to reduce, arrest, or delay any of the symptoms of the disease (biochemical, histologic and/or behavioral). Administration of a therapeutic can reduce or eliminate mild cognitive impairment in patients that have not yet developed characteristic Alzheimer's pathology.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

The antibodies of the application can be prepared as pharmaceutical compositions containing a therapeutically effective amount of the antibody as an active ingredient in a pharmaceutically acceptable carrier. The carrier can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They can be sterilized by conventional, well-known sterilization techniques (e.g., filtration). The compositions can contain pharmaceutically acceptable auxiliary substances as required to approximate physiological conditions such as pH adjusting and buffering agents, stabilizing, thickening, lubricating and coloring agents, etc. The concentration of the antibodies of the application in such pharmaceutical formulation can vary widely, i.e., from less than about 0.5%, usually at or at least about 1% to as much as 15 or 20% by weight and will be selected primarily based on required dose, fluid volumes, viscosities, etc., according to the particular mode of administration selected.

The mode of administration for therapeutic use of the antibodies of the application can be any suitable route that delivers the agent to the host. For example, the compositions described herein can be formulated to be suitable for parenteral administration, e.g., intradermal, intramuscular, intraperitoneal, intravenous, subcutaneous, intranasal or intracranial administration, or they can be administered into the cerebrospinal fluid of the brain or spine.

The treatment can be given in a single dose schedule, or as a multiple dose schedule in which a primary course of treatment can be with 1-10 separate doses, followed by other doses given at subsequent time intervals required to maintain and or reinforce the response, for example, at 1-4 months for a second dose, and if needed, a subsequent dose(s) after several months. Examples of suitable treatment schedules include: (i) 0, 1 month and 6 months, (ii) 0, 7 days and 1 month, (iii) 0 and 1 month, (iv) 0 and 6 months, or other schedules sufficient to elicit the desired responses expected to reduce disease symptoms or reduce severity of disease.

The antibodies of the application can be lyophilized for storage and reconstituted in a suitable carrier prior to use. This technique has been shown to be effective with antibody and other protein preparations and art-known lyophilization and reconstitution techniques can be employed.

According to particular embodiments, a composition used in the treatment of a tauopathy can be used in combination with other agents that are effective for treatment of related neurodegenerative diseases. In the case of AD, antibodies of the application can be administered in combination with agents that reduce or prevent the deposition of amyloid-beta (Abeta). It is possible that PHF-tau and Abeta pathologies are synergistic. Therefore, combination therapy targeting the clearance of both PHF-tau and Abeta and Abeta-related pathologies at the same time can be more effective than targeting each individually. In the case of Parkinson's Disease and related neurodegenerative diseases, immune modulation to clear aggregated forms of the alpha-synuclein protein is also an emerging therapy. A combination therapy which targets the clearance of both tau and alpha-synuclein proteins simultaneously can be more effective than targeting either protein individually.

In another general aspect, the application relates to a method of producing a pharmaceutical composition comprising a monoclonal antibody or antigen-binding fragment thereof of the application, comprising combining a monoclonal antibody or antigen-binding fragment thereof with a pharmaceutically acceptable carrier to obtain the pharmaceutical composition.

### Diagnostic Methods and Kits

Monoclonal anti-PHF-tau antibodies of the application can be used in methods of diagnosing AD or other tauopathies in a subject.

Thus, in another general aspect, the application relates to methods of detecting the presence of PHF-tau in a subject and methods of diagnosing tauopathies in a subject by detecting the presence of PHF-tau in the subject using a monoclonal antibody or antigen-binding fragment thereof of the application.

Phosphorylated tau can be detected in a biological sample from a subject (e.g., blood, serum, plasma, interstitial fluid, or cerebral spinal fluid sample) by contacting the biological sample with the diagnostic antibody reagent and detecting binding of the diagnostic antibody reagent to phosphorylated tau in the sample from the subject. Assays for carrying out the detection include well-known methods such as ELISA, immunohistochemistry, western blot, or in vivo imaging.

Diagnostic antibodies or similar reagents can be administered by intravenous injection into the body of the patient, or directly into the brain by any suitable route that delivers the agent to the host. The dosage of antibody should be within the same ranges as for treatment methods. Typically, the antibody is labeled, although in some methods, the primary antibody with affinity for phosphorylated tau is unlabeled, and a secondary labeling agent is used to bind to the primary antibody. The choice of label depends on the means of detection. For example, a fluorescent label is suitable for optical detection. Use of paramagnetic labels is suitable for tomographic detection without surgical intervention. Radioactive labels can also be detected using PET or SPECT.

Diagnosis is performed by comparing the number, size, and/or intensity of labeled PHF-tau, tau aggregates, and/or neurofibrillary tangles in a sample from the subject or in the subject, to corresponding baseline values. The baseline values can represent the mean levels in a population of healthy individuals. Baseline values can also represent previous levels determined in the same subject.

The diagnostic methods described above can also be used to monitor a subject's response to therapy by detecting the presence of phosphorylated tau in a subject before, during or after the treatment. A decrease in values relative to baseline signals a positive response to treatment. Values can also increase temporarily in biological fluids as pathological tau is being cleared from the brain.

The present application is further directed to a kit for performing the above described diagnostic and monitoring methods. Typically, such kits contain a diagnostic reagent such as the antibodies of the application, and optionally a detectable label. The diagnostic antibody itself can contain the detectable label (e.g., fluorescent molecule, biotin, etc.) which is directly detectable or detectable via a secondary reaction (e.g., reaction with streptavidin). Alternatively, a second reagent containing the detectable label cab be used, where the second reagent has binding specificity for the primary antibody. In a diagnostic kit suitable for measuring PHF-tau in a biological sample, the antibodies of the kit can be supplied pre-bound to a solid phase, such as to the wells of a microtiter dish.

The contents of all cited references (including literature references, issued patents, published patent applications, and co-pending patent applications) cited throughout this application are hereby expressly incorporated by reference.

### EMBODIMENTS

The application provides also the following non-limiting embodiments.

Embodiment 1 is an isolated monoclonal antibody or antigen-binding fragment thereof that binds to a tau protein at an epitope of the tau protein having or within the amino acid sequence of SEQ ID NO: 1, wherein the antibody or antigen-binding fragment thereof binds paired helical filament (PHF)-tau, preferably human PHF-tau.

Embodiment 1a is an isolated monoclonal antibody or antigen-binding fragment thereof that binds to a tau protein at an epitope of the tau protein consisting of or within the amino acid sequence of SEQ ID NO: 1, wherein the antibody or antigen-binding fragment thereof binds paired helical filament (PHF)-tau, preferably human PHF-tau.

Embodiment 2 is the isolated monoclonal antibody or antigen-binding fragment thereof of embodiment 1 or 1a, wherein:
(a) the epitope of the tau protein comprises either one of phosphorylated S433 or phosphorylated S435 of the tau protein, but does not comprise phosphorylated S433 and phosphorylated S435;
(b) the epitope of the tau protein comprises one or more of phosphorylated T427, phosphorylated S433 and phosphorylated S435 of the tau protein, but does not comprise all of phosphorylated T427, phosphorylated S433 and phosphorylated S435;
(c) the epitope of the tau protein comprises one or more of phosphorylated T427 and phosphorylated S433 of the tau protein, but does not comprise phosphorylated S435, and does not comprise all of phosphorylated T427, phosphorylated S433 and phosphorylated S435; or
(d) the epitope of the tau protein comprises phosphorylated T427 of the tau protein, but does not comprise phosphorylated S433 or phosphorylated S435.

Embodiment 3 is the isolated monoclonal antibody or antigen-binding fragment thereof of any one of embodiments 1-2, wherein the monoclonal antibody or antigen-binding fragment thereof comprises:
(a) immunoglobulin heavy chain HCDR1, HCDR2 and HCDR3 having the polypeptide sequences of SEQ ID NOs: 4, 5 and 6, respectively; and immunoglobulin light chain LCDR1, LCDR2 and LCDR3 having the polypeptide sequences of SEQ ID NOs: 7, 8 and 9, respectively;
(b) immunoglobulin heavy chain HCDR1, HCDR2 and HCDR3 having the polypeptide sequences of SEQ ID NOs: 14, 15 and 16, respectively; and immunoglobulin light chain LCDR1, LCDR2 and LCDR3 having the polypeptide sequences of SEQ ID NOs: 17, 18 and 19, respectively;
(c) immunoglobulin heavy chain HCDR1, HCDR2 and HCDR3 having the polypeptide sequences of SEQ ID NOs: 24, 25 and 26, respectively; and immunoglobulin light chain LCDR1, LCDR2 and LCDR3 having the polypeptide sequences of SEQ ID NOs: 27, 18 and 19, respectively;
(d) immunoglobulin heavy chain HCDR1, HCDR2 and HCDR3 having the polypeptide sequences of SEQ ID NOs: 32, 33 and 34, respectively; and immunoglobulin light chain LCDR1, LCDR2 and LCDR3 having the polypeptide sequences of SEQ ID NOs: 17, 18 and 35, respectively; or
(e) immunoglobulin heavy chain HCDR1, HCDR2 and HCDR3 having the polypeptide sequences of SEQ ID NOs: 40, 41 and 42, respectively; and immunoglobulin light chain LCDR1, LCDR2 and LCDR3 having the polypeptide sequences of SEQ ID NOs: 17, 18 and 43, respectively.

Embodiment 3a is the isolated monoclonal antibody or antigen-binding fragment thereof of any one of embodiments 1-3, wherein the monoclonal antibody or antigen-binding fragment thereof comprises:
(a) immunoglobulin heavy chain HCDR1, HCDR2 and HCDR3 consisting of the polypeptide sequences of SEQ ID NOs: 4, 5 and 6, respectively; and immunoglobulin light chain LCDR1, LCDR2 and LCDR3 consisting of the polypeptide sequences of SEQ ID NOs: 7, 8 and 9, respectively;
(b) immunoglobulin heavy chain HCDR1, HCDR2 and HCDR3 consisting of the polypeptide sequences of SEQ ID NOs: 14, 15 and 16, respectively; and immunoglobulin light chain LCDR1, LCDR2 and LCDR3 consisting of the polypeptide sequences of SEQ ID NOs: 17, 18 and 19, respectively;
(c) immunoglobulin heavy chain HCDR1, HCDR2 and HCDR3 consisting of the polypeptide sequences of SEQ ID NOs: 24, 25 and 26, respectively; and immunoglobulin light chain LCDR1, LCDR2 and LCDR3 consisting of the polypeptide sequences of SEQ ID NOs: 27, 18 and 19, respectively;
(d) immunoglobulin heavy chain HCDR1, HCDR2 and HCDR3 consisting of the polypeptide sequences of SEQ ID NOs: 32, 33 and 34, respectively; and immunoglobulin light chain LCDR1, LCDR2 and LCDR3 consisting of the polypeptide sequences of SEQ ID NOs: 17, 18 and 35, respectively; or
(e) immunoglobulin heavy chain HCDR1, HCDR2 and HCDR3 consisting of the polypeptide sequences of SEQ ID NOs: 40, 41 and 42, respectively; and immunoglobulin light chain LCDR1, LCDR2 and LCDR3 consisting of the polypeptide sequences of SEQ ID NOs: 17, 18 and 43, respectively.

Embodiment 4 is the isolated monoclonal antibody or antigen-binding fragment thereof of any one of embodiments 1-3a, comprising a heavy chain variable region having a polypeptide sequence at least 90% identical to SEQ ID NO: 2, 12, 22, 30 or 38, or a light chain variable region having a polypeptide sequence at least 90% identical to SEQ ID NO: 3, 13, 23, 31 or 39.

Embodiment 5 is the isolated monoclonal antibody or antigen-binding fragment thereof of any one of embodiments 1-4, comprising a heavy chain variable region having a polypeptide sequence of SEQ ID NO: 2, 12, 22, 30 or 38, or a light chain variable region having a polypeptide sequence of SEQ ID NO: 3, 13, 23, 31 or 39.

Embodiment 5a is the isolated monoclonal antibody or antigen-binding fragment thereof of any one of embodiments 1-5, comprising a heavy chain variable region consisting of a polypeptide sequence of any one of SEQ ID NO: 2, 12, 22, 30 or 38, or a light chain variable region consisting of a polypeptide sequence of any one of SEQ ID NO: 3, 13, 23, 31 or 39.

Embodiment 6 is the isolated monoclonal antibody or antigen-binding fragment thereof of any one of embodiments 1-5a, comprising:
(a) a heavy chain variable region having the polypeptide sequence of SEQ ID NO: 2, and a light chain variable region having the polypeptide sequence of SEQ ID NO: 3;
(b) a heavy chain variable region having the polypeptide sequence of SEQ ID NO: 12, and a light chain variable region having the polypeptide sequence of SEQ ID NO: 13;
(c) a heavy chain variable region having the polypeptide sequence of SEQ ID NO: 22, and a light chain variable region having the polypeptide sequence of SEQ ID NO: 23;
(d) a heavy chain variable region having the polypeptide sequence of SEQ ID NO: 30, and a light chain variable region having the polypeptide sequence of SEQ ID NO: 31; or
(e) a heavy chain variable region having the polypeptide sequence of SEQ ID NO: 38, and a light chain variable region having the polypeptide sequence of SEQ ID NO: 39.

Embodiment 6a is the isolated monoclonal antibody or antigen-binding fragment thereof of any one of embodiments 1-6, comprising:
(a) a heavy chain variable region consisting of the polypeptide sequence of SEQ ID NO: 2, and a light chain variable region consisting of the polypeptide sequence of SEQ ID NO: 3;
(b) a heavy chain variable region consisting of the polypeptide sequence of SEQ ID NO: 12, and a light chain variable region consisting of the polypeptide sequence of SEQ ID NO: 13;
(c) a heavy chain variable region consisting of the polypeptide sequence of SEQ ID NO: 22, and a light chain variable region consisting of the polypeptide sequence of SEQ ID NO: 23;
(d) a heavy chain variable region consisting of the polypeptide sequence of SEQ ID NO: 30, and a light chain variable region consisting of the polypeptide sequence of SEQ ID NO: 31; or
(e) a heavy chain variable region consisting of the polypeptide sequence of SEQ ID NO: 38, and a light chain variable region consisting of the polypeptide sequence of SEQ ID NO: 39.

Embodiment 7 is the isolated monoclonal antibody or antigen-binding fragment thereof of any one of embodiments 1-6a, comprising:
(a) a heavy chain having the polypeptide sequence of SEQ ID NO: 10, and a light chain having the polypeptide sequence of SEQ ID NO: 11;
(b) a heavy chain having the polypeptide sequence of SEQ ID NO: 20, and a light chain having the polypeptide sequence of SEQ ID NO: 21;
(c) a heavy chain having the polypeptide sequence of SEQ ID NO: 28, and a light chain having the polypeptide sequence of SEQ ID NO: 29;
(d) a heavy chain having the polypeptide sequence of SEQ ID NO: 36, and a light chain having the polypeptide sequence of SEQ ID NO: 37; or
(e) a heavy chain having the polypeptide sequence of SEQ ID NO: 44, and a light chain having the polypeptide sequence of SEQ ID NO: 45.

Embodiment 7a is the isolated monoclonal antibody or antigen-binding fragment thereof of any one of embodiments 1-7, comprising:
(a) a heavy chain consisting of the polypeptide sequence of SEQ ID NO: 10, and a light chain consisting of the polypeptide sequence of SEQ ID NO: 11;
(b) a heavy chain consisting of the polypeptide sequence of SEQ ID NO: 20, and a light chain consisting of the polypeptide sequence of SEQ ID NO: 21;
(c) a heavy chain consisting of the polypeptide sequence of SEQ ID NO: 28, and a light chain consisting of the polypeptide sequence of SEQ ID NO: 29;
(d) a heavy chain consisting of the polypeptide sequence of SEQ ID NO: 36, and a light chain consisting of the polypeptide sequence of SEQ ID NO: 37; or
(e) a heavy chain consisting of the polypeptide sequence of SEQ ID NO: 44, and a light chain consisting of the polypeptide sequence of SEQ ID NO: 45.

Embodiment 8 is the isolated monoclonal antibody or antigen-binding fragment thereof of any one of embodiments 1-7a, comprising:
(a) an immunoglobulin heavy chain HCDR1, HCDR2 and HCDR3 having the polypeptide sequences of SEQ ID NOs: 4, 5 and 6, respectively; and immunoglobulin light chain LCDR1, LCDR2 and LCDR3 having the polypeptide sequences of SEQ ID NOs: 7, 8 and 9, respectively;
(b) a heavy chain variable region having the polypeptide sequence of SEQ ID NO: 2, and a light chain variable region having the polypeptide sequence of SEQ ID NO: 3; or
(c) a heavy chain having the polypeptide sequence of SEQ ID NO: 10, and a light chain having the polypeptide sequence of SEQ ID NO: 11.

Embodiment 9 is the isolated monoclonal antibody or antigen-binding fragment thereof of any one of embodiments 1-7a, comprising:
(a) an immunoglobulin heavy chain HCDR1, HCDR2 and HCDR3 having the polypeptide sequences of SEQ ID NOs: 14, 15 and 16, respectively; and immunoglobulin light chain LCDR1, LCDR2 and LCDR3 having the polypeptide sequences of SEQ ID NOs: 17, 18 and 19, respectively;
(b) a heavy chain variable region having the polypeptide sequence of SEQ ID NO: 12, and a light chain variable region having the polypeptide sequence of SEQ ID NO: 13; or
(c) a heavy chain having the polypeptide sequence of SEQ ID NO: 20, and a light chain having the polypeptide sequence of SEQ ID NO: 21.

Embodiment 10 is an isolated nucleic acid encoding the monoclonal antibody or antigen-binding fragment thereof of any one of embodiments 1 to 9.

Embodiment 11 is a vector comprising the isolated nucleic acid of embodiment 10.

Embodiment 12 is a host cell comprising the isolated nucleic acid of embodiment 10.

Embodiment 13 is a pharmaceutical composition comprising the isolated monoclonal antibody or antigen-binding fragment thereof of any one of embodiments 1 to 9 and a pharmaceutically acceptable carrier.

Embodiment 14 is a method of blocking tau seeding in a subject in need thereof, comprising administering to the subject the pharmaceutical composition of embodiment 13.

Embodiment 15 is a method of treating a tauopathy in a subject in need thereof, comprising administering to the subject the pharmaceutical composition of embodiment 13.

Embodiment 16 is a method of reducing pathological tau aggregation or spreading of tauopathy in a subject in need thereof, comprising administering to the subject the pharmaceutical composition of embodiment 13.

Embodiment 17 is the method of embodiment 15 or 16, wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

Embodiment 17a is the method of any one of embodiments 15-17, further comprising administering to the subject an additional agent for treating the tauopathy in the subject in need thereof.

Embodiment 18 is a method of producing the monoclonal antibody or antigen-binding fragment thereof of any one of embodiments 1 to 9, comprising culturing a cell comprising a nucleic acid encoding the monoclonal antibody or antigen-binding fragment thereof under conditions to produce the monoclonal antibody or antigen-binding fragment thereof and recovering the monoclonal antibody or antigen-binding fragment thereof from the cell or cell culture.

Embodiment 19 is a method of detecting the presence of PHF-tau in a biological sample from a subject, comprising contacting the biological sample with the isolated monoclonal antibody or antigen-binding fragment thereof of any one of embodiments 1 to 9, and detecting binding of the monoclonal antibody or antigen-binding fragment thereof to PHF-tau in the sample from the subject.

Embodiment 20 is the method of embodiment 19, wherein the biological sample is a blood, serum, plasma, interstitial fluid, or cerebral spinal fluid sample.

Embodiment 21 is a method of producing a pharmaceutical composition comprising the monoclonal antibody or antigen-binding fragment thereof of any one of embodiments 1 to 9, comprising combining the monoclonal antibody or antigen-binding fragment thereof with a pharmaceutically acceptable carrier to obtain the pharmaceutical composition.

Embodiment 22 is an isolated monoclonal antibody or antigen-binding fragment thereof of any one of embodiments 1 to 9 for use in treating a tauopathy, in a subject in need thereof.

Embodiment 23 is an isolated monoclonal antibody or antigen-binding fragment thereof of any one of embodiments 1 to 9 or the pharmaceutical composition of embodiment 13 for use in treating a tauopathy, such as familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, or dementia pugulistica (boxing disease), in a subject in need thereof.

Embodiment 24 is a use of an isolated monoclonal antibody or antigen-binding fragment thereof of any one of embodiments 1 to 9 for manufacturing a medicament in treating a tauopathy in a subject in need thereof.

Embodiment 25 is a use of an isolated monoclonal antibody or antigen-binding fragment thereof of any one of embodiments 1 to 9 for manufacturing a medicament for treating a tauopathy, such as familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, or dementia pugulistica (boxing disease), in a subject in need thereof.

Embodiment 26 is a method of diagnosing a tauopathy in a subject by detecting the presence of PHF-tau in a biological sample from the subject, comprising contacting the biological sample with the isolated monoclonal antibody or antigen-binding fragment thereof of any one of embodiments 1 to 9, and detecting binding of the antibody or antigen-binding fragment to PHF-tau in the sample from the subject.

### EXAMPLES

The following examples of the application are to further illustrate the nature of the invention. It should be understood that the following examples do not limit the application and that the scope of the application is to be determined by the appended claims.

### Example 1—Antibody Generation

Anti-PHF-tau (PT/53, PT/66, PT/69, PT/81) and anti-in vitro aggregated tau antibodies (hTau/60) were generated using standard hybridoma technology in Tau knockout (KO) mice (Kohler and Milstein Nature 256:495-7, 1975). Obtained hybridomas were seeded in 96-well plates and screened after 10 days in a direct ELISA on 25 ng/well coated PHF-tau as described below. Positive cells were tested for cross-reactivity on 10 ng/well coated with control tau (SEQ ID NO: 51) expressed in E. Coli BL21 cells and purified by heat treatment and ammonium sulphate precipitation. PT/53, PT/66, PT/69, PT/81 and hTau60 were found to bind to both PHF tau and control tau (SEQ ID NO: 51). PT/66, PT/69 and hTau/60 were prioritized for V-region cloning and humanization.

Positive cells were immediately subcloned and positive clones were frozen in liquid nitrogen. All hybridomas were grown in Dulbecco's modified Eagle's medium supplemented with 10% fetal calf serum (Hyclone, Europe), Hybridoma Fusion Cloning Supplement (2%) (Roche, Brussels, Belgium), 2% HT (Sigma, USA), 1 mM sodium pyruvate, 2 mM L-glutamine and penicillin (100 U/ml), and Streptomycin (50 mg/ml).

Antibody variable regions were cloned from select hybridoma cells onto mouse IgG1/IgG2/κ background and expressed and purified using routine methods. Briefly, hybridoma cells were lysed in RLT Buffer (Qiagen catalog #79216) and frozen at −70° C. The lysate was thawed at 37° C. and RNA was isolated using RNeasy 96 Kit (Qiagen catalog #74182).

An aliquot of RNA was used to synthesize cDNA using a gene specific reverse primer mix using primers designed to anneal to the constant region for mouse IgG heavy chain, mouse Kappa light chain and mouse Lambda light chain.

An aliquot of cDNA was used in PCR reactions with mouse primer sets designed to amplify either IgG heavy chain variable regions, kappa light chain variable regions or lambda light chain variable regions. The forward primers consisted of multiple primers designed to anneal to Framework 1 and the reverse primer was designed to anneal to the constant region. An aliquot of the PCR products was run on a 2% agarose gel and the heavy and kappa PCR products showed a visible band of correct size.

The heavy chain and kappa light chain PCR products were sequenced (Sanger method) using a heavy chain or kappa light chain reverse primer designed to anneal to the respective constant region. The sequences were analyzed and aligned to identify the closest matching mouse germline. The first ten amino acids of the heavy and kappa chain Framework 1 sequence were replaced using the matching germline sequence. The IgG heavy chain and kappa variable region amino acid sequences were codon optimized and synthesized. The codon optimized IgG heavy chain and kappa light chain variable regions were synthesized and cloned the fragments into a mouse IgG2a isotype heavy chain and kappa light chain isotype expression vectors.

Antibody variable regions were cloned from selected hybridoma cells, sequenced using standard methods, and subcloned into expression vectors for mAb and Fab. Mab was produced on a mouse IgG2a/K background and expressed and purified by affinity chromatography (protein A). Fab was produced as chimeric versions with the mouse variable domains fused to human IgG1/κ constant domains and a His tag at the C-terminus of the heavy chain. Fab was transiently expressed in HEK293F cells and purified by affinity chromatography (HisTrap).

### Example 2—Antibody Characterization

### ELISA and Western Blotting

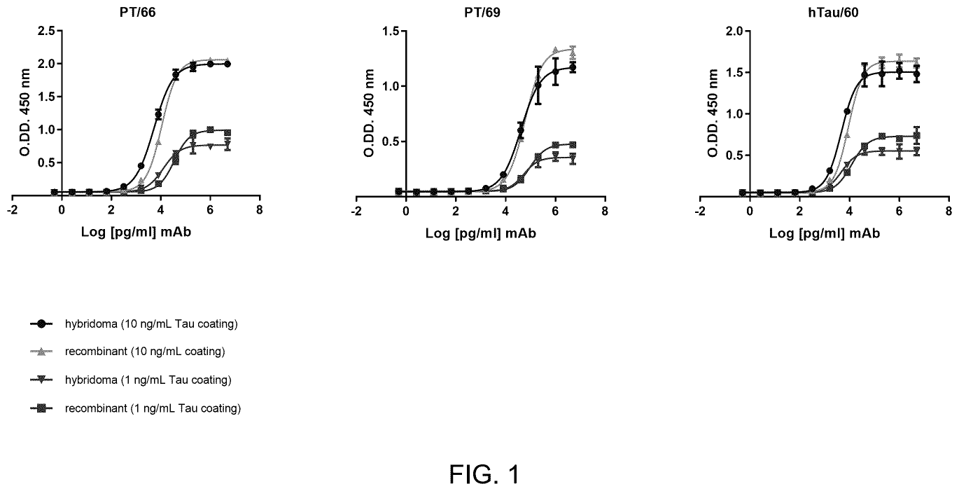

Binding to recombinant WT (2N4R) tau was analyzed by ELISA. Full-length Tau protein (1 ng/mL or 10 ng/mL) was directly coated to the plate and incubated with different concentrations of either recombinantly- or hybridoma-produced PT/66, PT/69 or hTau/60 antibodies (FIG. 1). As expected, the lower coating concentrations of Tau resulted in lower maximal values. No substantial difference was observed between binding profiles of recombinant and hybridoma produced antibodies.

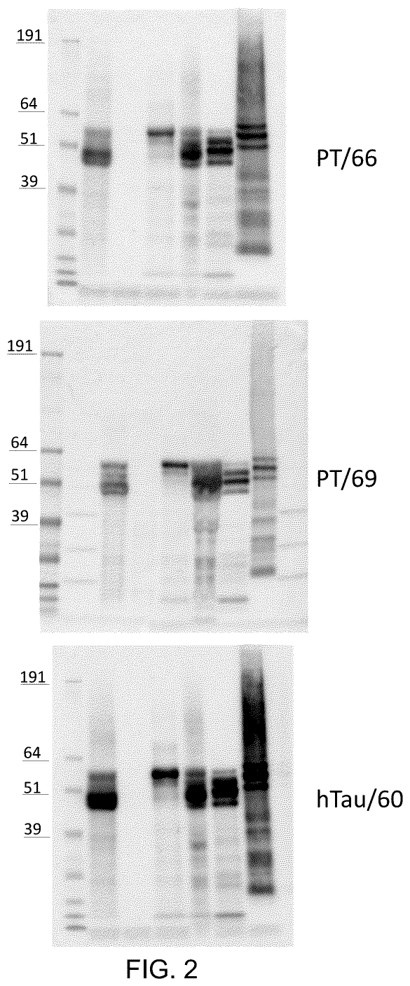

Further profiling of antibodies was performed by evaluating their binding to tau in brain samples from different species (mouse, dog, monkey, and human). For human tau, a distinction was made between soluble Tau (heat-stable extract from non-AD human brain) and aggregated PHF Tau (sarcosyl insoluble prep from human AD brain). To be able to detect lower affinity interactions to non-Tau related proteins, antibodies were tested at 1 μg/mL, and relatively high amounts of brain homogenates (20 μg of total protein) were loaded on the gel. An overview of these profiles is shown in FIG. 2.

### Binding Assessment by Surface Plasmon Resonance (SPR)

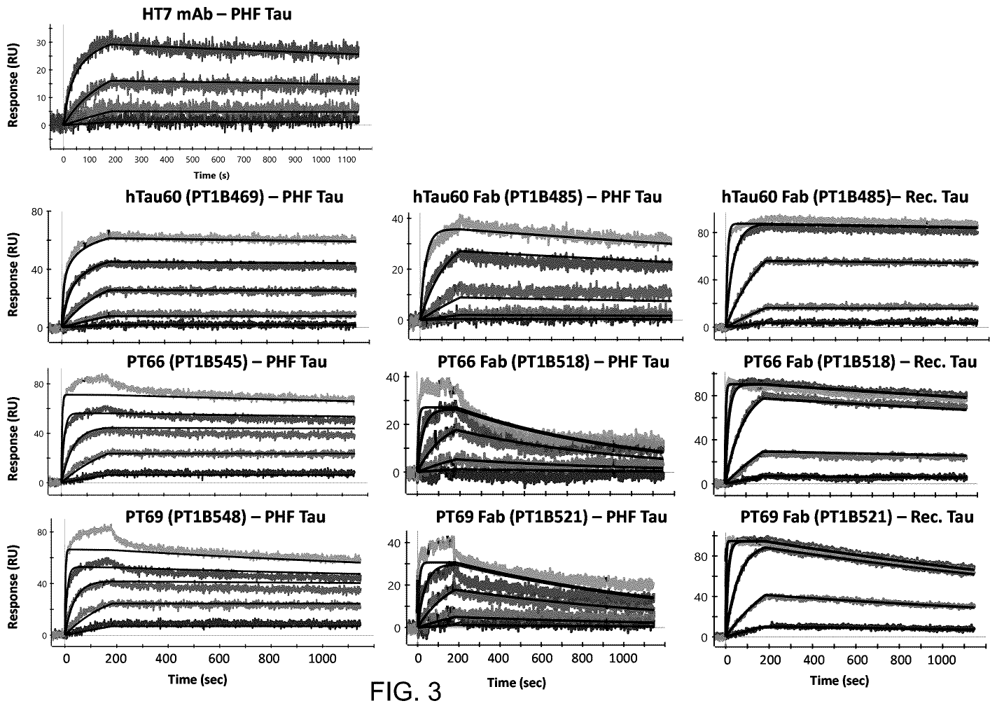

The interactions with PHF-tau and recombinant tau were assessed by SPR on ProteOn (Bio-Rad, Hercules, CA) instrument for PT66, PT69, hTau60 anti-tau antibodies and their corresponding Fab fragments. Because of the multimeric/aggregated nature of PHF-tau with multiple copies of the epitope, and the bivalent nature of IgGs, mAb affinity was influenced by avidity in this study format. Fab affinity provides information on the intrinsic affinity of the antibody. Representative sensorgrams of mAbs and their Fab fragments binding to PHF Tau are shown in FIG. 3 and a summary is represented in Table 1 below. HT7 was used as a reference.

TABLE 1
SPR binding affinities of hTau60, PT66, PT69 and
their Fab fragments to PHF and recombinant Tau

| | PHF-tau | | Rec. Tau |
|---|---|---|---|
| | mAb K_{D} | Fab K_{D} | Fab K_{D} |
| Antibody name | (pM)* | (pM) | (pM) |
| HT7 (reference mAb) | 291 | n.a | n.a |
| hTau60 (PT1B469) | 69 | 427 | 26 |
| PT66 (PT1B545) | 56 | 514 | 40 |
| PT69 (PT1B548) | 102 | 437 | 69 |

*Reported affinities should be treated as apparent affinities due to bivalent nature of IgGs and multimeric/aggregated nature of PHF-tau
n.a = not available
Rec. = recombinant

### Antibody Profiling Using IHC

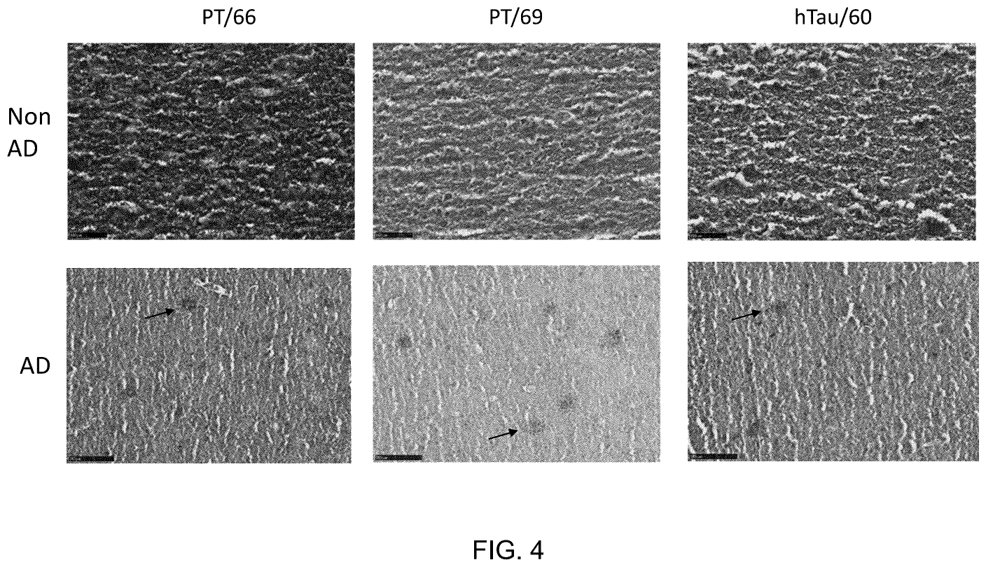

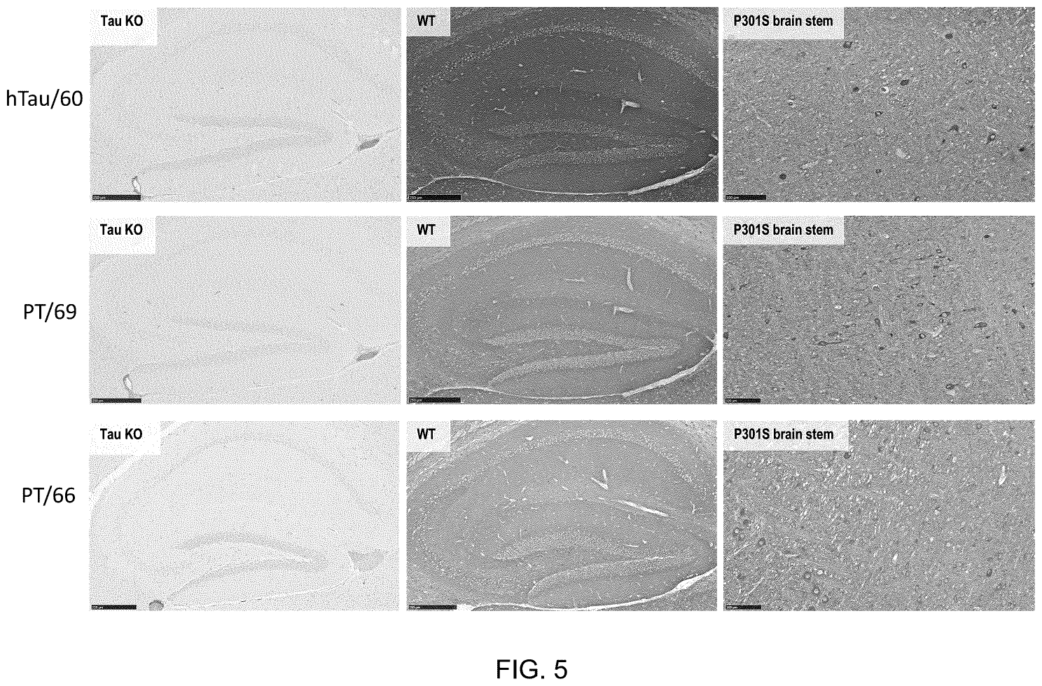

Immunohistochemical analysis was performed on cryosections of AD and non-AD brain to confirm reactivity with physiological and pathophysiological tau in situ. PT66, PT69/PT87 and hTau60 showed strong binding to both soluble (non-AD and AD brain) and aggregated tau (AD brain) (FIG. 4). Additional IHC analysis was performed on formalin-fixed paraffin-embedded tissue from WT, Tau knock-out (KO), and P301S (5 month) mouse brain. Lack of signal in brain slices from Tau KO mice confirmed the specificity of the antibodies. Non-aggregated tau was detected in sections from WT and P301S mice while aggregated tau in brainstem of P301S mice was also stained by PT/66, PT/69 and hTau/60 (FIG. 5).

### Example 3—Functional Testing in Cellular Assays

PT/66, PT/69 and hTau/60 were tested for inhibition of tau seeding in an immunodepletion assay which makes use of HEK cells expressing two chromophore-tagged K18 tau fragments that generate a signal when in close proximity by aggregation. When the cells are treated with seeds of aggregated and phosphorylated full-size tau derived from different sources, a K18 aggregate is induced that can be quantified by counting fluorescence resonance energy transfer (FRET)-positive cells using fluorescence-activated cell sorting (FACS) (Holmes et al., 2014, PNAS. 111(41): E4376-85). Biochemical analysis of the immunodepleted samples was done by a hTau60/hTau60 self-sandwich MSD assay.

To investigate if the maximum percentage inhibition value is related to the density of epitopes on the seeds or to the number of seeds that contain the PT/66, PT/69 and hTau/60 epitope, immunodepletion assays were performed. AD tau seeds were incubated with test antibody and removed from the solution with protein G beads. The depleted supernatant was tested for residual seeding capacity in the chromophore-K18-containing HEK cells and analyzed by FACS as previously described (Holmes et al., PNAS. 111(41): E4376-85, 2014).

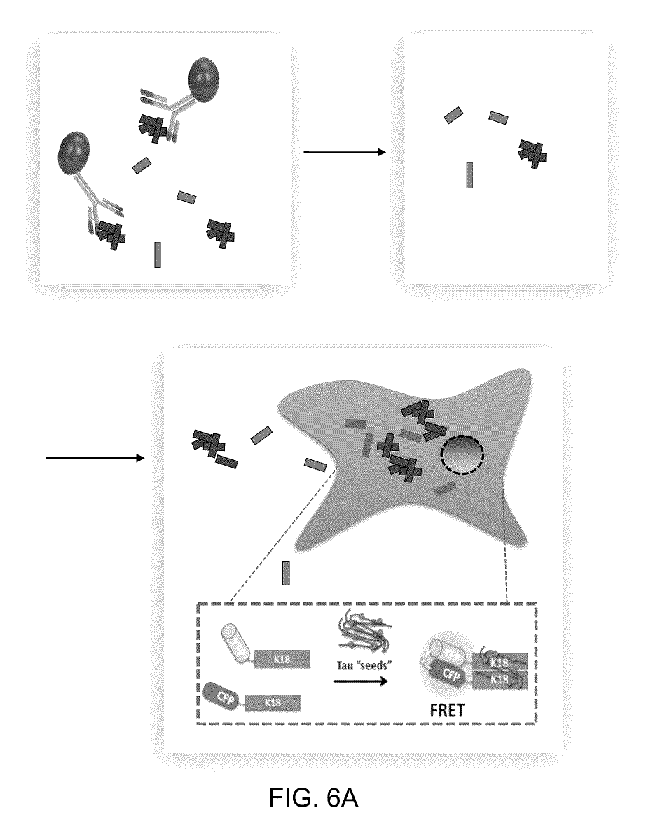

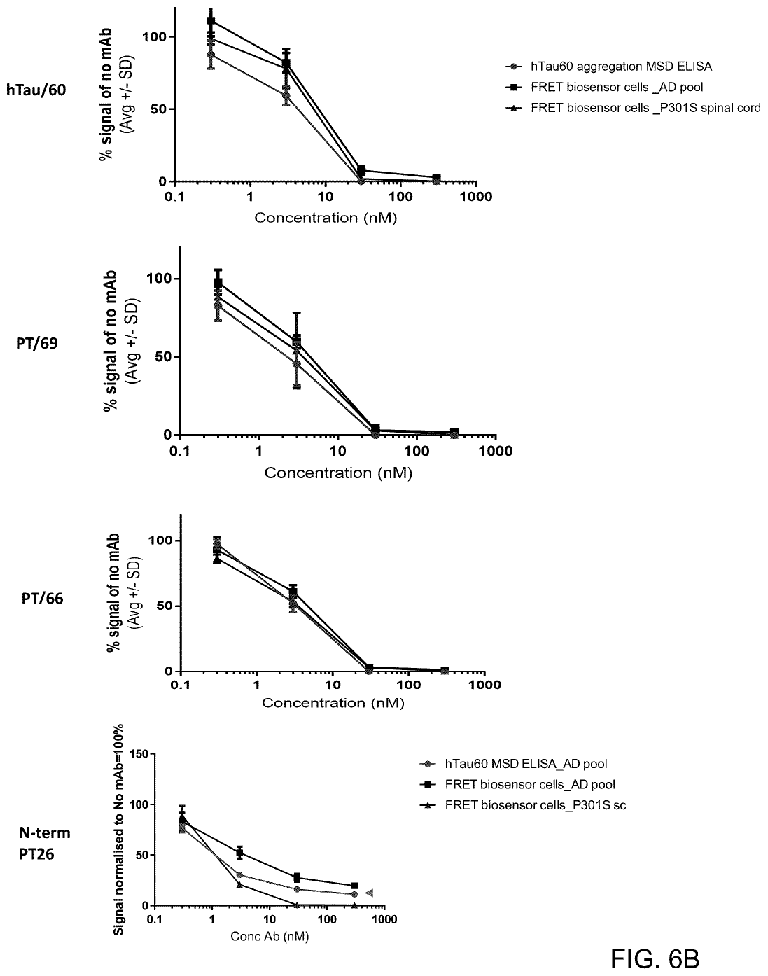

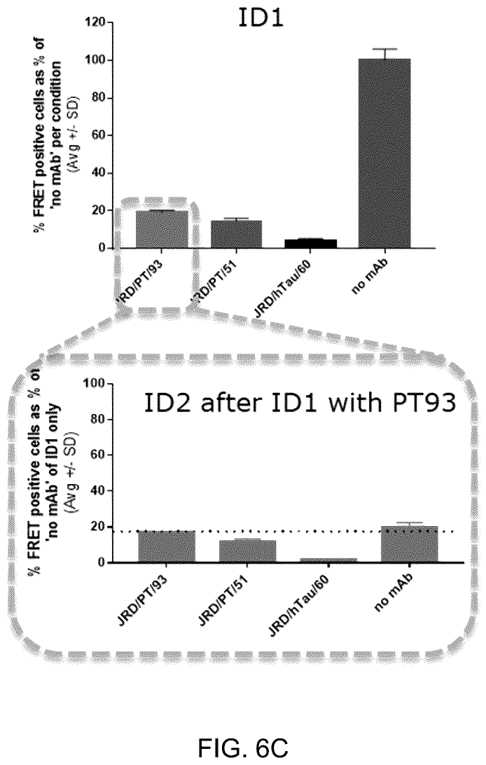

Homogenates containing tau seeds for immunodepletion were generated from spinal cords from 22- to 23-week old P301S transgenic animals or from cryopreserved human AD brain tissue. In the human AD brain immunodepletion assay, the supernatant after depletion was tested in the presence of the transfection reagent Lipofectamine2000 to obtain an acceptable assay window. The tau seeding (and hTau60/hTau60 aggregation signal) could be reduced completely with C-terminal antibodies, but not with the N-terminal antibody PT93 (which has been described in Vandermeeren et al., J Alzheimers Dis, 2018; 65(1):265-281, the relevant content of which is incorporated herein by reference), in total homogenates from human AD brain and in spinal cord homogenates from P301S transgenic mice, (>95% inhibition; FIGS. 6A-B and Table 2, showing % inhibition in comparison to negative control, average of at least 2 independent experiments).

TABLE 2
Immunodepletions in the cellular assays

| | | | Immunodepletion |
|---|---|---|---|
| | | Immunodepletion | FRET biosensor |
| | Conc. | FRET biosensor | cells _P301S |
| Antibody | (nM) | cells _AD pool | spinal cord |
| PT66 | 3 | 38.6 ± 4.8 | 46.5 ± 4.5 |
| | 300 | 98.6 ± 0.6 | 99.7 ± 0.2 |
| PT69 | 3 | 40.3 ± 4.2 | 45.8 ± 24.1 |
| | 300 | 97.9 ± 1.6 | 99.8 ± 0.1 |
| hTau60 | 3 | 17.7 ± 6.7 | 22 ± 13.7 |
| | 300 | 97.2 ± 1.1 | 99.7 ± 0.05 |
| PT26 | 3 | 47.6 ± 5.9 | 79.1 ± 2.1 |
| | 300 | 80.4 ± 2.1 | 99.5 ± 0.2 |

In a separate experiment, it was shown that in a sequential immunodepletion assay, after the initial immunodepletion with the N-terminal antibody PT93, the C-terminal antibody hTau60 further depleted all remaining PHF aggregates, while no or some further depletion was observed with PT93 or PT51 (HT7-like) (which is described in Vandermeeren et al., J Alzheimers Dis, 2018; 65(1):265-281, the relevant content of which is incorporated herein by reference), respectively (FIG. 6C).

The mechanism of action for tau antibody therapy is still a matter of debate and multiple mechanisms have been proposed. Antibody-mediated clearance of extracellular seeds by microglial cells has recently been suggested as one dominant mechanism of action (Funk et al., J Biol Chem. 290(35):21652-62, 2015 and McEwan et al., 2017, PNAS 114:574-9). In this context, immunodepletion of human-brain-derived seeding material can be considered the most translational cellular result, and the high efficacy of the C-terminal antibodies PT66, PT68/PT87 and hTau60 in this type of cellular assay suggests that the HFA versions of these antibodies will be effective therapeutics.

### Example 4—In Vivo Efficacy of PT/66, PT/69 and hTau/60 in the ePHF Injection Model

### Introduction

To evaluate tau antibody efficacy in vivo, mice displaying brain tau pathology are essential model systems (Julien et al., Methods Mol Biol. 849:473-91, 2012). Several of these models have been described, and they can generally be divided in three groups: 1) tau transgenic mice overexpressing WT or mutant (e.g., P301L or P301S) tau with the mutants showing severe pathology after 5-9 months, depending on the strain (Allen et al., J Neurosci. 22(21):9340-51, 2002; Scattoni et al., Behav Brain Res. 208(1):250-7, 2010; Terwel et al., J Biol Chem. 280(5):3963-73, 2005; Yoshiyama et al., Neuron. 53(3):337-51, 2007) mice with spatio-temporally-regulated expression of mutant tau (e.g., P301L) (Liu et al., Brain Imaging Behav. 6(4):610-20, 2012) or a pro-aggregating fragment (e.g., K18) (Mocanu et al., J Neurosci. 28(3):737-48, 2008); and 3) mice with expression of both mutant tau and APP displaying both plaque and tau pathologies (Oddo et al., J Neurochem. 102(4):1053-63, 2007).

While mice expressing mutant tau develop a strong pathology, the onset of pathology can vary between animals, causing variability in studies, and the relative contribution of cell-autonomous tau aggregation and spreading to the overall tau aggregation signal is not clear. Therefore, models that can be used to effectively study tau seeding and spreading (e.g., de Calignon et al., 2012, Neuron. 73(4):685-97, 2012; Liu et al., Id.) are of high value. The translational value of such models is further strengthened by the finding that injection of ALZ17 mice (a strain expressing normal human tau) with brain homogenates derived from different tauopathies induces the formation of tau inclusions with a morphology that resembles tauopathy in the human brain. For example, injection of mice with material from Argyrophilic grain disease samples resulted in deposits with a spheroid or comma-like structure characteristic of the disease itself, and AD-like tau pathology was observed in mice injected with AD material (Clavaguera et al., 2013, PNAS 110(23):9535-40).

Thus, a transgenic P301L mouse injection model has been established, wherein a pro-aggregating fragment of tau, such as synthetic K18 fibrils (Li and Lee, Biochemistry. 45(51):15692-701, 2006) or PFH-tau seeds derived from human AD brain, is injected in cortical or hippocampal regions of P301L transgenic mouse models at an age at which cell-autonomous aggregation has not started. The injection model aims to mimic the critical extracellular seeding component of tau spreading. The injected K18 or PHF-tau seed induces tauopathy at the injection site and, to a lesser degree, at the connected contralateral region (Peeraer et al., Neurobiol Dis. 73:83-95, 2015). The model enables testing of the anti-seeding potential of antibodies, such as anti-tau antibodies of the application, when co-injected with the AD-brain-derived PHF-tau seeds or the K18 fibrils (Iba et al., 2015, J Neurosci. 33(3):1024-37, 2013; Iba et al., Acta Neuropathol. 130(3):349-62).

Cortical injection of a sarcosyl-insoluble fraction of post-mortem AD brain triggers a slowly progressing increase of tau aggregation. In the injected hemisphere, the first signals are measured 1 month after injection and progress further 3 months after injection. Five months after injection, some animals start to form tangles driven by the P301L mutation (Terwel et al., 2005, Id.). AT8 staining levels increase between 1 and 3 months (U.S. Pat. No. 10,766,953), so antibody efficacy experiments are analyzed 2 months after co-injection. Additionally, hippocampal injection of a sarcosyl-insoluble fraction of post-mortem AD brain triggers a dose-dependent progressing increase of tau aggregation measured by MesoScale Discoveries (MSD) analysis of sarcosyl insoluble fractions from the injected hemispheres.

### Animal Treatment and Intracranial Injections

For injection studies, transgenic tau-P301L mice, expressing the longest human tau isoform with the P301L mutation (tau-4R/2N-P301L) (Terwel et al., 2005, Id.) were used for surgery at the age of 3 months. All experiments were performed in compliance with protocols approved by the local ethical committee. For stereotactic surgery, the mice received a unilateral (right hemisphere) injection in the hippocampus (AP −2.0, ML+2.0 (from bregma), DV 1.8 mm (from dura)) 3 μl (speed 0.25 μl/min) with a sarcosyl insoluble prep from postmortem AD tissue (enriched paired helical filaments, ePHF) in the presence or absence of monoclonal antibodies. Mice were sacrificed for dissection (2 months after intracranial injection).

### Extraction Procedure

Mouse tissue from the injected hemisphere was weighed and homogenized in 6 volumes of homogenization buffer (10 mM Tris HCl (pH7.6); 0.8 M NaCl; 10% w/v sucrose; 1 mM EGTA; PhosStop phosphatase inhibitor cocktail; complete EDTA-free mini protease inhibitors). The homogenate was centrifuged at 28,000×g for 20 minutes, and 1% N-lauroylsarcosine was added after taking an aliquot from the resulting supernatant (total homogenate). After 90 minutes (900 rpm, 37° C.), the solutions were again centrifuged at 184,000×g for 1 hour. The supernatants were kept as sarcosyl-soluble fraction, whereas the pellet containing the sarcosyl-insoluble material was resuspended in homogenization buffer.

### Biochemical Analysis

Coating antibody (AT8) was diluted in PBS (1 μg/ml) and aliquoted into MSD plates (30 μL per well) (L15XA, Mesoscale Discoveries), which were incubated overnight at 4° C. After washing with 5×200 μl of PBS/0.5% Tween-20, the plates were blocked with 0.1% casein in PBS and washed again with 5×200 μl of PBS/0.5% Tween-20. After adding samples and standards (both diluted in 0.1% casein in PBS), the plates were incubated overnight at 4° C. Subsequently, the plates were washed with 5×200 μl of PBS/0.5% Tween-20, and SULFO-TAG™ conjugated detection antibody (AT8) in 0.1% casein in PBS was added and incubated for 2 hours at room temperature while shaking at 600 rpm. After a final wash (5×200 μl of PBS/0.5% Tween-20), 150 μl of 2×buffer T was added, and plates were read with an MSD imager. Raw signals were normalized against a standard curve consisting of 16 dilutions of a sarcosyl insoluble prep from postmortem AD brain (ePHF) and were expressed as arbitrary units (AU) ePHF. Statistical analysis (ANOVA with Bonferroni post-test) was performed with the GraphPad prism software.

### Results

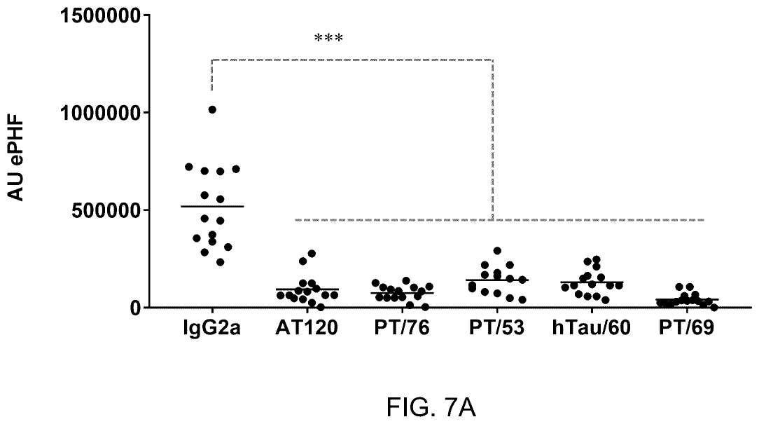

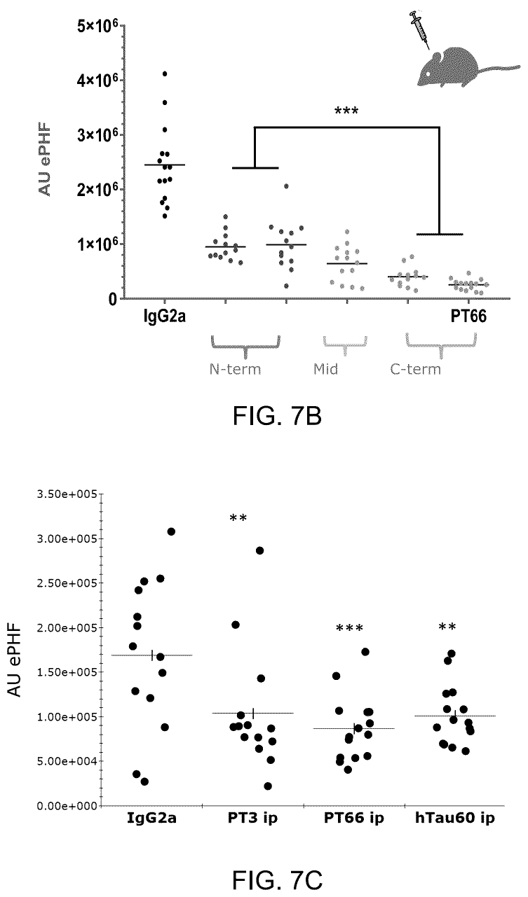

Several of the internal anti-Tau antibodies had been evaluated in this co-injection model (see, e.g., U.S. Pat. No. 10,766,953 and Vandermeeren et al., J. Alzheimers Dis. 65(1):265-81 (2018)). Activity of these antibodies (recombinantly expressed as IgG2a) under the hippocampal co-injection model were compared according to Table 3 below. Co-injection of Tau antibodies attenuated ePHF-induced tau aggregation in P301L mice (FIG. 7A). AT120 is described in Vandermeeren et al., J Alzheimers Dis, 2018; 65(1):265-281, the relevant content of which is incorporated herein by reference, and it binds to the proline-rich domain (PRD) of Tau. PT/76 is described Vandermeeren et al., J Alzheimers Dis, 2018; 65(1):265-281, the relevant content of which is incorporated herein by reference, and it binds close to the microtubule-ninding domain (MTBD) in tau.

TABLE 3
ePHF co-injection groups

| | | pmole | pmole | |
|---|---|---|---|---|
| Group | epitope | ePHF | Ab | n |
| IgG | Non Tau | 0.6 | 4.5 | 15 |
| AT120 | PRD | 0.6 | 4.5 | 15 |
| hTau/60 | C-terminal | 0.6 | 4.5 | 15 |
| PT/69 | C-terminal | 0.6 | 4.5 | 15 |
| PT/53 | C-terminal | 0.6 | 4.5 | 14 |
| PT/76 | MTBD | 0.6 | 4.5 | 15 |

Data are summarized in Table 4.

TABLE 4
Summary of results from the co-injection studies

| | | % inhibition | |
|---|---|---|---|
| Antibody | Epitope | to control | P-value* |
| AT120 | mid-term | 82 | <0001 |
| PT/76 | MTBD | 86 | <0001 |
| PT/53 | C-term | 73 | <0001 |
| PT/69 | C-term | 92 | <0001 |
| hTau/60 | C-term | 75 | <0001 |

*One way ANOVA using Bonferroni correction for multiple testing.

In separate experiments, the activity of C-terminal antibodies PT66 and PT81 in the hippocampal co-injection mouse model was compared to the activity of internal Janssen anti-tau antibodies that bind to the N-terminal portion and the middle portion of PHF Tau. Antibodies were co-injected with ePHF tau (0.6 pmoles) into the cortex. Co-injection of C-terminal antibodies attenuated ePHF-induced tau aggregation in P301L mice significantly more than other antibodies (FIG. 7B).

In a follow-up study, efficacy of PT66, hTau60 and PT3 (see U.S. Pat. No. 10,633,435, the contents of which are incorporated herein by reference) were compared upon peripheral dosing (20 mg/kg; 2×/week) of each antibody after intracranial co-injection of antibody+PHF. The peripheral dosing started 2 weeks before intracranial injections of PHF and continued during the life phase of the experiment. Consistent with the first study, co-administration of each of PT66, hTau60 and PT3 reduced the ePHF-induced aggregation signal and inhibited the seeding induced by ePHF (FIG. 7C).

### Example 5—Epitope Mapping of C-Terminal Antibodies

### Materials and Methods.

### Synthesis of Array Peptides

To reconstruct epitopes of the target molecule, a library of peptides (20-mers with an overlap of 18 amino acids) covering the Tau 441 sequence was synthesized. An amino functionalized polypropylene support was obtained by grafting with a proprietary hydrophilic polymer formulation, followed by reaction with t-butyloxycarbonyl-hexamethylenediamine (BocHMDA) using dicyclohexylcarbodiimide (DCC) with N-hydroxybenzotriazole (HOBt), and subsequent cleavage of the Boc-groups using trifluoroacetic acid (TFA). Standard Fmoc-peptide synthesis was used to synthesize peptides on the amino-functionalized solid support by custom modified JANUS liquid handling stations (Perkin Elmer). Synthesis of structural mimics was done using Pepscan's proprietary Chemically Linked Peptides on Scaffolds (CLIPS) technology. CLIPS technology allows to structure peptides into single loops, double loops, triple loops, sheet-like folds, helix-like folds, and combinations thereof. CLIPS templates are coupled to cysteine residues. The side-chains of multiple cysteines in the peptides are coupled to one or two CLIPS templates. For example, a 0.5 mM solution of the P2 CLIPS (2,6-bis(bromomethyl)pyridine) is dissolved in ammonium bicarbonate (20 mM, pH 7.8)/acetonitrile (1:3 (v/v)). This solution is added onto the peptide arrays. The CLIPS template will bind to side-chains of two cysteines as present in the solid-phase bound peptides of the peptide-arrays (455 wells plate with 3 μl wells). The peptide arrays are gently shaken in the solution for 30 to 60 minutes while completely covered in solution. Finally, the peptide arrays are washed extensively with excess of H₂O and sonicated in disrupt-buffer containing 1% SDS/0.1% beta-mercaptoethanol in PBS (pH 7.2) at 70° C. for 30 minutes, followed by sonication in H₂O for another 45 minutes. The T3 CLIPS carrying peptides were made in a similar way but now with three cysteines.

### Elisa Screening

The binding of antibodies (recombinantly expressed as IgG2a) to each of the synthesized peptides was tested in a pepscan-based ELISA. The peptide arrays were incubated with primary antibody solution (overnight at 4° C.). After washing, the peptide arrays were incubated with a 1/1000 dilution of an appropriate antibody peroxidase conjugate (SBA; Table 4) for one hour at 25° C. After washing, the peroxidase substrate 2,2′-azino-di-3-ethylbenzthiazoline sulfonate (ABTS) and 20 μl/ml of 3 percent H₂O₂ were added. After one hour, the color development was measured. The color development was quantified with a charge coupled device (CCD) camera and an image processing system.

### Results

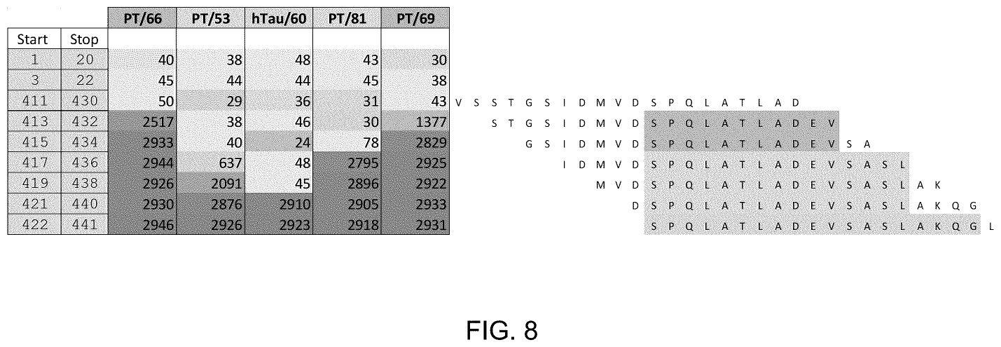

Data show binding of five related antibodies, PT/66, PT/53, hTau60, PT/81 and PT/69, to a series of peptides starting from 1 until residue 441 (FIG. 8). For convenient interpretation, only the first 2 N-terminal peptides and a series of peptides from 411 until 441 are shown. For these antibodies, no binding to other Tau peptides (i.e., other locations on Tau) was observed. Detailed mapping is shown in Table 5 below.

TABLE 5
Epitope mapping

| | | SEQ ID | Tolerated | Non-tolerated |
|---|---|---|---|---|
| Antibody | Epitope | NO: | phospho | phospho |
| hTau60 | ₄₃₁EVSASLAKQG₄₄₀ | 52 | S433, S435 | S433 + S435 |
| PT50/PT53 | ₄₂₆ATLADEVSASLAK₄₃₈ | 53 | T427, S433 | S435, |
| | | | | T427 + S433 + S435 |
| PT66 | ₄₂₆ATLADEVSASL₄₃₆ | 54 | T427, S433, | T427 + S433 + S435 |
| | | | S435 | |
| PT69/PT87 | ₄₂₂SPQLATLADEVSASLAK₄₃₈ | 55 | T427, S433 | S435, |
| | | | | T427 + S433 + S435 |
| PT81 | ₄₂₈LADEVSASL₄₃₆ | 56 | T427 | S433, S435, |
| | | | | T427 + S433 + S435 |

### Example 6—Epitope Processing

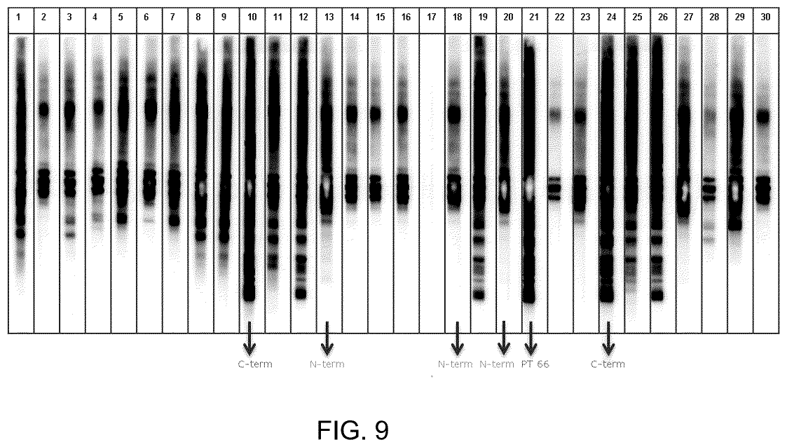

While not wishing to be bound by theories, it is believed that the improved efficacy by a C-terminal antibody, such as PT66, and the lower efficacy by N-terminal antibodies, can be explained by extensive processing of epitopes at the N-terminus of PHF-tau. This is based on the Western blot profile obtained by the analysis of a human AD brain derived PHF sample with a Western blotting screen (FIG. 9). In this experiment, a large amount of sample was loaded on a 1-well gel. After blotting, individual strips were incubated with a panel of anti-tau mAbs binding to different epitopes. The strips of lanes 10, 21 and 24 were incubated with C-terminal tau mAbs, which showed extensive staining of lower molecular weight bands. In comparison, the strips of lanes 13, 18 and 20 were incubated with N-terminal tau mAbs, which did not show extensive staining of lower molecular weight bands. Further rationalization is described in (Vandermeeren et al., 2018, J Alzheimers Dis, 2018; 65(1):265-281).

While embodiments of the invention have been described in detail, and with reference to specific embodiments thereof, it will be apparent to one of ordinary skill in the art that various changes and modifications can be made therein without departing from the spirit and scope of the invention.

SEQUENCES

| SEQ ID | | |
|---|---|---|
| NO: | Description | Sequence |
| 1 | Tau epitope | SPQLATLADEVSASLAK |
| | (aa422-438) | |
| 2 | hTau60 VH | EVQLQQSGPELVKPGTSVKISCKVFGYTFTDYYMNWVKQSHGKSLEWIGDINPDNGET |
| | | TYNQKFKGKATLTVDKSSSTAYMELRSLTSEDSAVYYCAKGATFVYWGQGTLVTVSA |
| 3 | hTau60 VL | DVVMTQTPLSLPVSLGDQASISCISSQSLVHSTGTTFLHWFLQKPGQSPKLLIYKVSNRF |
| | | SGVPDRFSGSGSGTDFTLKISRVEAEDLGFYFCSQSTYFPLTFGSGTKLEIK |
| 4 | hTau60 HCDR1 | GYTFTDYYMN |
| 5 | hTau60 HCDR2 | DINPDNGETTYNQKFKG |
| 6 | hTau60 HCDR3 | GATFVY |
| 7 | hTau60 LCDR1 | ISSQSLVHSTGTTFLH |
| 8 | hTau60 LCDR2 | KVSNRFS |
| 9 | hTau60 LCDR3 | SQSTYFPLT |
| 10 | hTau60 HC | EVQLQQSGPELVKPGTSVKISCKVFGYTFTDYYMNWVKQSHGKSLEWIGDINPDNGET |
| | | TYNQKFKGKATLTVDKSSSTAYMELRSLTSEDSAVYYCAKGATFVYWGQGTLVTVSA |
| | | AKTTAPSVYPLAPVCGDTTGSSVTLGCLVKGYFPEPVTLTWNSGSLSSGVHTFPAVLQS |
| | | DLYTLSSSVTVTSSTWPSQSITCNVAHPASSTKVDKKIEPRGPTIKPCPPCKCPAPNLLGG |
| | | PSVFIFPPKIKDVLMISLSPIVTCVVVDVSEDDPDVQISWFVNNVEVHTAQTQTHREDYN |
| | | STLRVVSALPIQHQDWMSGKEFKCKVNNKDLPAPIERTISKPKGSVRAPQVYVLPPPEE |
| | | EMTKKQVTLTCMVTDFMPEDIYVEWTNNGKTELNYKNTEPVLDSDGSYFMYSKLRVE |
| | | KKNWVERNSYSCSVVHEGLHNHHTTKSFSRTPGK |
| 11 | hTau60 LC | DVVMTQTPLSLPVSLGDQASISCISSQSLVHSTGTTFLHWFLQKPGQSPKLLIYKVSNRF |
| | | SGVPDRFSGSGSGTDFTLKISRVEAEDLGFYFCSQSTYFPLTFGSGTKLEIKRADAAPTV |
| | | SIFPPSSEQLTSGGASVVCFLNNFYPKDINVKWKIDGSERQNGVLNSWTDQDSKDSTYS |
| | | MSSTLTLTKDEYERHNSYTCEATHKTSTSPIVKSFNRNEC |
| 12 | PT/66 VH | QVQLQQPGAELVKPGASVKMSCKASGYTFTSYWITWVKQRPGQGLEWIGDIHPGRGS |
| | | TKSNEKFKSKATLTVDTSSSTAYMQFSSLTSEDSAVYYCARRWGFDYWGQGTTLTVSS |
| 13 | PT/66 VL | DIVITQDELSNPVTSGESVSISCRSSKSLLYKDGKTYLNWFLQRPGQSPQLLIYLMSTRA |
| | | SGVSDRFSGSGSGTDFTLEISRVKAEDVGVYYCQQLVDYPLTFGAGTKLELK |
| 14 | PT/66 HCDR1 | GYTFTSYWIT |
| 15 | PT/66 HCDR2 | DIHPGRGSTKSNEKFKS |
| 16 | PT/66 HCDR3 | RWGFDY |
| 17 | PT/66 LCDR1 | RSSKSLLYKDGKTYLN |
| 18 | PT/66 LCDR2 | LMSTRAS |
| 19 | PT/66 LCDR3 | QQLVDYPLT |
| 20 | PT/66 HC | QVQLQQPGAELVKPGASVKMSCKASGYTFTSYWITWVKQRPGQGLEWIGDIHPGRGS |
| | | TKSNEKFKSKATLTVDTSSSTAYMQFSSLTSEDSAVYYCARRWGFDYWGQGTTLTVSS |
| | | AKTTAPSVYPLAPVCGDTTGSSVTLGCLVKGYFPEPVTLTWNSGSLSSGVHTFPAVLQS |
| | | DLYTLSSSVTVTSSTWPSQSITCNVAHPASSTKVDKKIEPRGPTIKPCPPCKCPAPNLLGG |
| | | PSVFIFPPKIKDVLMISLSPIVTCVVVDVSEDDPDVQISWFVNNVEVHTAQTQTHREDYN |
| | | STLRVVSALPIQHQDWMSGKEFKCKVNNKDLPAPIERTISKPKGSVRAPQVYVLPPPEE |
| | | EMTKKQVTLTCMVTDFMPEDIYVEWTNNGKTELNYKNTEPVLDSDGSYFMYSKLRVE |
| | | KKNWVERNSYSCSVVHEGLHNHHTTKSFSRTPGK |
| 21 | PT/66 LC | DIVITQDELSNPVTSGESVSISCRSSKSLLYKDGKTYLNWFLQRPGQSPQLLIYLMSTRA |
| | | SGVSDRFSGSGSGTDFTLEISRVKAEDVGVYYCQQLVDYPLTFGAGTKLELKRADAAP |
| | | TVSIFPPSSEQLTSGGASVVCFLNNFYPKDINVKWKIDGSERQNGVLNSWTDQDSKDST |
| | | YSMSSTLTLTKDEYERHNSYTCEATHKTSTSPIVKSFNRNEC |
| 22 | PT/69 VH | QVQLQQPGAELVKPGASVKMSCKASGYTFTNYWITWVKQRPGQGLEWIGDIYPGSGR |
| | | TKSNEKFKNKATLTADTSSSTAYMQLSSLTSEDSAVYYCTRRWGLDYWGQGTTLTVS |
| | | S |
| 23 | PT/69 VL | DIVITQDELSNPVTSGESVSISCRSNKSLLYKDGKTYLNWFLQRPGQSPQLLIYLMSTRA |
| | | SGVSDRFSGSGSGTDFTLEISRVKAEDVGVYYCQQLVDYPLTFGAGTKLELK |
| 24 | PT/69 HCDR1 | GYTFTNYWIT |
| 25 | PT/69 HCDR2 | DIYPGSGRTKSNEKFKN |
| 26 | PT/69 HCDR3 | RWGLDY |
| 27 | PT/69 LCDR1 | RSNKSLLYKDGKTYLN |
| 18 | PT/69 LCDR2 | LMSTRAS |
| 19 | PT/69 LCDR3 | QQLVDYPLT |
| 28 | PT/69 HC | QVQLQQPGAELVKPGASVKMSCKASGYTFTNYWITWVKQRPGQGLEWIGDIYPGSGR |
| | | TKSNEKFKNKATLTADTSSSTAYMQLSSLTSEDSAVYYCTRRWGLDYWGQGTTLTVS |
| | | SAKTTAPSVYPLAPVCGDTTGSSVTLGCLVKGYFPEPVTLTWNSGSLSSGVHTFPAVLQ |
| | | SDLYTLSSSVTVTSSTWPSQSITCNVAHPASSTKVDKKIEPRGPTIKPCPPCKCPAPNLLG |
| | | GPSVFIFPPKIKDVLMISLSPIVTCVVVDVSEDDPDVQISWFVNNVEVHTAQTQTHREDY |
| | | NSTLRVVSALPIQHQDWMSGKEFKCKVNNKDLPAPIERTISKPKGSVRAPQVYVLPPPE |
| | | EEMTKKQVTLTCMVTDFMPEDIYVEWTNNGKTELNYKNTEPVLDSDGSYFMYSKLRV |
| | | EKKNWVERNSYSCSVVHEGLHNHHTTKSFSRTPGK |
| 29 | PT/69 LC | DIVITQDELSNPVTSGESVSISCRSNKSLLYKDGKTYLNWFLQRPGQSPQLLIYLMSTRA |
| | | SGVSDRFSGSGSGTDFTLEISRVKAEDVGVYYCQQLVDYPLTFGAGTKLELKRADAAP |
| | | TVSIFPPSSEQLTSGGASVVCFLNNFYPKDINVKWKIDGSERQNGVLNSWTDQDSKDST |
| | | YSMSSTLTLTKDEYERHNSYTCEATHKTSTSPIVKSFNRNEC |
| 30 | PT/53 VH | EVKLMESGGGLVQPGASLRLSCAASGFTFTDYYMSWVRQPPGKAPEWLALIRNKANG |
| | | YTTKYAASVKGRFTISRDNSQNILYLQMNTLRAEDSATYYCVKAVWFAYWGQGTLVT |
| | | VSA |
| 31 | PT/53 VL | DIVITQDELSNPVTSGESVSISCRSSKSLLYKDGKTYLNWFLQRPGQSPQLLIYLMSTRA |
| | | SGVSDRFSGSGSGTDFTLEISRVKAEDVGVYYCLQLVEYPYTFGGGTKLEIK |
| 32 | PT/53 HCDR1 | GFTFTDYYMS |
| 33 | PT/53 HCDR2 | LIRNKANGYTTKYAASVKG |
| 34 | PT/53 HCDR3 | AVWFAY |
| 17 | PT/53 LCDR1 | RSSKSLLYKDGKTYLN |
| 18 | PT/53 LCDR2 | LMSTRAS |
| 35 | PT/53 LCDR3 | LQLVEYPYT |
| 36 | PT/53 HC | EVKLMESGGGLVQPGASLRLSCAASGFTFTDYYMSWVRQPPGKAPEWLALIRNKANG |
| | | YTTKYAASVKGRFTISRDNSQNILYLQMNTLRAEDSATYYCVKAVWFAYWGQGTLVT |
| | | VSAAKTTAPSVYPLAPVCGDTTGSSVTLGCLVKGYFPEPVTLTWNSGSLSSGVHTFPAV |
| | | LQSDLYTLSSSVTVTSSTWPSQSITCNVAHPASSTKVDKKIEPRGPTIKPCPPCKCPAPNL |
| | | LGGPSVFIFPPKIKDVLMISLSPIVTCVVVDVSEDDPDVQISWFVNNVEVHTAQTQTHRE |
| | | DYNSTLRVVSALPIQHQDWMSGKEFKCKVNNKDLPAPIERTISKPKGSVRAPQVYVLPP |
| | | PEEEMTKKQVTLTCMVTDFMPEDIYVEWTNNGKTELNYKNTEPVLDSDGSYFMYSKL |
| | | RVEKKNWVERNSYSCSVVHEGLHNHHTTKSFSRTPGK |
| 37 | PT/53 LC | DIVITQDELSNPVTSGESVSISCRSSKSLLYKDGKTYLNWFLQRPGQSPQLLIYLMSTRA |
| | | SGVSDRFSGSGSGTDFTLEISRVKAEDVGVYYCLQLVEYPYTFGGGTKLEIKRADAAPT |
| | | VSIFPPSSEQLTSGGASVVCFLNNFYPKDINVKWKIDGSERQNGVLNSWTDQDSKDSTY |
| | | SMSSTLTLTKDEYERHNSYTCEATHKTSTSPIVKSFNRNEC |
| 38 | PT/81 VH | QVQLQQPGAELVRPGASVILSCKASGYTFTNSWIHWVKQRPGRVLEWIGRIDPNSGGT |
| | | RYNENFKSKATLTVDKPSSTAYMQLSSLTSEDSAVYFCSRGVLHDYWGQGTTLTVSS |
| 39 | PT/81 VL | DIVITQDELSNPVTSGESVSISCRSSKSLLYKDGKTYLNWFLQRPGQSPQLLIYLMSTRA |
| | | SGVSDRFSVSGSGTDFTLEISRVQAEDVGVYYCQQLVEYPYTFGGGTKLEIK |
| 40 | PT/81 HCDR1 | GYTFTNSWIH |
| 41 | PT/81 HCDR2 | RIDPNSGGTRYNENFKS |
| 42 | PT/81 HCDR3 | GVLHDY |
| 17 | PT/81 LCDR1 | RSSKSLLYKDGKTYLN |
| 18 | PT/81 LCDR2 | LMSTRAS |
| 43 | PT/81 LCDR3 | QQLVEYPYT |
| 44 | PT/81 HC | QVQLQQPGAELVRPGASVILSCKASGYTFTNSWIHWVKQRPGRVLEWIGRIDPNSGGT |
| | | RYNENFKSKATLTVDKPSSTAYMQLSSLTSEDSAVYFCSRGVLHDYWGQGTTLTVSSA |
| | | KTTAPSVYPLAPVCGDTTGSSVTLGCLVKGYFPEPVTLTWNSGSLSSGVHTFPAVLQSD |
| | | LYTLSSSVTVTSSTWPSQSITCNVAHPASSTKVDKKIEPRGPTIKPCPPCKCPAPNLLGGP |
| | | SVFIFPPKIKDVLMISLSPIVTCVVVDVSEDDPDVQISWFVNNVEVHTAQTQTHREDYNS |
| | | TLRVVSALPIQHQDWMSGKEFKCKVNNKDLPAPIERTISKPKGSVRAPQVYVLPPPEEE |
| | | MTKKQVTLTCMVTDFMPEDIYVEWTNNGKTELNYKNTEPVLDSDGSYFMYSKLRVE |
| | | KKNWVERNSYSCSVVHEGLHNHHTTKSFSRTPGK |
| 45 | PT/81 LC | DIVITQDELSNPVTSGESVSISCRSSKSLLYKDGKTYLNWFLQRPGQSPQLLIYLMSTRA |
| | | SGVSDRFSVSGSGTDFTLEISRVQAEDVGVYYCQQLVEYPYTFGGGTKLEIKRADAAPT |
| | | VSIFPPSSEQLTSGGASVVCFLNNFYPKDINVKWKIDGSERQNGVLNSWTDQDSKDSTY |
| | | SMSSTLTLTKDEYERHNSYTCEATHKTSTSPIVKSFNRNEC |
| 46 | Tau isoform | MAEPRQEFEVMEDHAGTYGLGDRKDQGGYTMHQDQEGDTDAGLKAEEAGIGDTPSL |
| | 0N3R | EDEAAGHVTQARMVSKSKDGTGSDDKKAKGADGKTKIATPRGAAPPGQKGQANATR |
| | | IPAKTPPAPKTPPSSGEPPKSGDRSGYSSPGSPGTPGSRSRTPSLPTPPTREPKKVAVVRTP |
| | | PKSPSSAKSRLQTAPVPMPDLKNVKSKIGSTENLKHQPGGGKVQIVYKPVDLSKVTSKC |
| | | GSLGNIHHKPGGGQVEVKSEKLDFKDRVQSKIGSLDNITHVPGGGNKKIETHKLTFREN |
| | | AKAKTDHGAEIVYKSPVVSGDTSPRHLSNVSSTGSIDMVDSPQLATLADEVSASLAKQ |
| | | GL |
| 47 | Tau isoform | MAEPRQEFEVMEDHAGTYGLGDRKDQGGYTMHQDQEGDTDAGLKESPLQTPTEDGS |
| | 1N3R | EEPGSETSDAKSTPTAEAEEAGIGDTPSLEDEAAGHVTQARMVSKSKDGTGSDDKKAK |
| | | GADGKTKIATPRGAAPPGQKGQANATRIPAKTPPAPKTPPSSGEPPKSGDRSGYSSPGSP |
| | | GTPGSRSRTPSLPTPPTREPKKVAVVRTPPKSPSSAKSRLQTAPVPMPDLKNVKSKIGST |
| | | ENLKHQPGGGKVQIVYKPVDLSKVTSKCGSLGNIHHKPGGGQVEVKSEKLDFKDRVQ |
| | | SKIGSLDNITHVPGGGNKKIETHKLTFRENAKAKTDHGAEIVYKSPVVSGDTSPRHLSN |
| | | VSSTGSIDMVDSPQLATLADEVSASLAKQGL |
| 48 | Tau isoform | MAEPRQEFEVMEDHAGTYGLGDRKDQGGYTMHQDQEGDTDAGLKESPLQTPTEDGS |
| | 2N3R | EEPGSETSDAKSTPTAEDVTAPLVDEGAPGKQAAAQPHTEIPEGTTAEEAGIGDTPSLED |
| | | EAAGHVTQARMVSKSKDGTGSDDKKAKGADGKTKIATPRGAAPPGQKGQANATRIPA |
| | | KTPPAPKTPPSSGEPPKSGDRSGYSSPGSPGTPGSRSRTPSLPTPPTREPKKVAVVRTPPK |
| | | SPSSAKSRLQTAPVPMPDLKNVKSKIGSTENLKHQPGGGKVQIVYKPVDLSKVTSKCGS |
| | | LGNIHHKPGGGQVEVKSEKLDFKDRVQSKIGSLDNITHVPGGGNKKIETHKLTFRENAK |
| | | AKTDHGAEIVYKSPVVSGDTSPRHLSNVSSTGSIDMVDSPQLATLADEVSASLAKQGL |
| 49 | Tau isoform | MAEPRQEFEVMEDHAGTYGLGDRKDQGGYTMHQDQEGDTDAGLKAEEAGIGDTPSL |
| | 0N4R | EDEAAGHVTQARMVSKSKDGTGSDDKKAKGADGKTKIATPRGAAPPGQKGQANATR |
| | | IPAKTPPAPKTPPSSGEPPKSGDRSGYSSPGSPGTPGSRSRTPSLPTPPTREPKKVAVVRTP |
| | | PKSPSSAKSRLQTAPVPMPDLKNVKSKIGSTENLKHQPGGGKVQIINKKLDLSNVQSKC |
| | | GSKDNIKHVPGGGSVQIVYKPVDLSKVTSKCGSLGNIHHKPGGGQVEVKSEKLDFKDR |
| | | VQSKIGSLDNITHVPGGGNKKIETHKLTFRENAKAKTDHGAEIVYKSPVVSGDTSPRHL |
| | | SNVSSTGSIDMVDSPQLATLADEVSASLAKQGL |
| 50 | Tau isoform | MAEPRQEFEVMEDHAGTYGLGDRKDQGGYTMHQDQEGDTDAGLKESPLQTPTEDGS |
| | 1N4R | EEPGSETSDAKSTPTAEDVTAPLVDEGAPGKQAAAQPHTEIPEGTTAEEAGIGDTPSLED |
| | | EAAGHVTQARMVSKSKDGTGSDDKKAKGADGKTKIATPRGAAPPGQKGQANATRIPA |
| | | KTPPAPKTPPSSGEPPKSGDRSGYSSPGSPGTPGSRSRTPSLPTPPTREPKKVAVVRTPPK |
| | | SPSSAKSRLQTAPVPMPDLKNVKSKIGSTENLKHQPGGGKVQIINKKLDLSNVQSKCGS |
| | | KDNIKHVPGGGSVQIVYKPVDLSKVTSKCGSLGNIHHKPGGGQVEVKSEKLDFKDRVQ |
| | | SKIGSLDNITHVPGGGNKKIETHKLTFRENAKAKTDHGAEIVYKSPVVSGDTSPRHLSN |
| | | VSSTGSIDMVDSPQLATLADEVSASLAKQGL |
| 51 | Tau isoform | MAEPRQEFEVMEDHAGTYGLGDRKDQGGYTMHQDQEGDTDAGLKESPLQTPTEDGS |
| | 2N4R | EEPGSETSDAKSTPTAEDVTAPLVDEGAPGKQAAAQPHTEIPEGTTAEEAGIGDTPSLED |
| | | EAAGHVTQARMVSKSKDGTGSDDKKAKGADGKTKIATPRGAAPPGQKGQANATRIPA |
| | | KTPPAPKTPPSSGEPPKSGDRSGYSSPGSPGTPGSRSRTPSLPTPPTREPKKVAVVRTPPK |
| | | SPSSAKSRLQTAPVPMPDLKNVKSKIGSTENLKHQPGGGKVQIINKKLDLSNVQSKCGS |
| | | KDNIKHVPGGGSVQIVYKPVDLSKVTSKCGSLGNIHHKPGGGQVEVKSEKLDFKDRVQ |
| | | SKIGSLDNITHVPGGGNKKIETHKLTFRENAKAKTDHGAEIVYKSPVVSGDTSPRHLSN |
| | | VSSTGSIDMVDSPQLATLADEVSASLAKQGL |

### REFERENCES

Abhinandan and Martin, Mol Immunol. 45:3832-9, 2008
Adams et al., Acta Crystallogr D Biol Crystallogr. 66(Pt 2):213-21, 2010
Allen et al., J Neurosci. 22(21):9340-51, 2002
Almagro, Mol Recognit. 17:132-43, 2004
Asuni et al., J Neurosci. 27:9115-29, 2007
Boutajangout et al., J Neurochem. 118:658-67, 2011
Boutajangout et al., J Neurosci. 30:16559-66, 2010
Brunden et al., Nat Rev Drug Discov. 8:783-93, 2009
Butner and Kirschner, J Cell Biol. 115(3):717-30, 1991
Chai et al., J Biol Chem. 286:34457-67, 2011
Chothia and Lesk, J Mol Biol. 196:901-17, 1987
Clavaguera et al., Nat Cell Biol. 11:909-13, 2009
Clavaguera et al., Proc Natl Acad Sci USA. 110(23):9535-40, 2013
Clavaguera et al., Proc Natl Acad Sci USA. 110(23):9535-40, 2013
Collaborative Computational Project, Number 4, Acta Crystallogr D Biol Crystallogr. 50(Pt 5):760-3, 1994
Collin et al., Brain. 137(Pt 10):2834-46, 2014
de Calignon et al., Neuron. 73(4):685-97, 2012
Emsley and Cowtan, Acta Crystallogr D Biol Crystallogr. 60(Pt 12 Pt 1):2126-32, 2004
Epitope Mapping Protocols in Methods in Molecular Biology, Vol. 66, G. E. Morris, Ed. (1996)
Fishwild et al., Nat Biotechnol. 14:845-51, 1996
Fransson et al., J Mol Biol. 398(2):214-31, 2010
Frost et al., J Biol Chem. 284:12845-52, 2009
Funk et al., J Biol Chem. 290(35):21652-62, 2015
Goedert et al., Biochemical J. 301(Pt3):871-877
Hanger et al., Trends Mol Med. 15:112-9, 2009
Hoffmann et al., Biochemistry. 36(26):8114-24, 1997
Holmes et al., Proc Natl Acad Sci USA. 111(41): E4376-85, 2014
Iba et al., Acta Neuropathol. 130(3):349-62, 2015
Iba et al., J Neurosci. 33(3):1024-37, 2013
Julien et al., Methods Mol Biol. 849:473-91, 2012
Kabsch, Acta Crystallogr D Biol Crystallogr. 66(Pt 2):125-32, 2010
Knappik et al., J Mol Biol. 296:57-86, 2000
Knight et al., Platelets. 15:409-18, 2004
Kohler and Milstein, Nature. 256:495-7, 1975
Krebs et al., J Immunol Methods. 254:67-84, 2001
Lee et al., Cell Rep. 16(6):1690-700, 2016
Lefranc et al., Dev Comp Immunol. 27:55-77, 2003
Leong et al., Cytokine. 16:106-19, 2001
Li and Lee, Biochemistry. 45(51):15692-701, 2006
Liu et al., Brain Imaging Behav. 6(4):610-20, 2012
Lonberg et al., Nature. 368:856-9, 1994
Malia et al., Proteins. 84:427-434, 2016
Martin and Thornton, J Mol Biol. 263(5):800-15, 1996
Matsuo et al., Neuron. 13(4):989-1002, 1994
McCoy et al., J Appl Crystallogr. 40(Pt 4):658-674, 2007
McEwan et al., 2017, PNAS 114(3):574-9
Mendez et al., Nat Genet. 15:146-56, 1997
Mercken et al., Acta Neuropathol. 84(3):265-72, 1992
Mercken, Ph.D. Thesis: University of Antwerp, Wilrijk-Antwerp, 1991
Mocanu et al., J Neurosci. 28(3):737-48, 2008
Morris et al., Nat Neurosci. 18(8):1183-9, 2015
Morris et al., Neuron, 70:410-26, 2011
Murshudov et al., Acta Crystallogr D Biol Crystallogr. 53(Pt 3):240-55, 1997
Oddo et al., J Neurochem. 102(4):1053-63, 2007
Otvos et al., J Neurosci Res. 39(6):669-73, 1994
Padlan et al., Mol. Immunol. 28:489-98, 1991
Peeraer et al., Neurobiol Dis. 73:83-95, 2015
Queen et al., Proc Natl Acad Sci USA. 86:10029-33, 1989
Scattoni et al., Behav Brain Res. 208(1):250-7, 2010
Schroeder et al., J Neuroimmune Pharmacol. 11(1):9-25, 2016
Seubert et al., J Biol Chem. 270(32):18917-22, 1995
Shi et al., J Mol Biol. 397:385-96, 2010
Strohl, Curr Opin Biotechnol. 20:685-91, 2009
Terwel et al., J Biol Chem. 280(5):3963-73, 2005
Wischik et al. Proc Natl Acad Sci USA. 85:4884-8, 1988
Wu and Kabat, J Exp Med. 132:211-50, 1970
Yang et al., Protein Eng. 16:761-70, 2003
Yoshiyama et al., Neuron. 53(3):337-51, 2007
Zhao et al., Protein Expr Purif. 67(2):182-9, 2009

1. An isolated monoclonal antibody or antigen-binding fragment thereof that binds to a tau protein at an epitope of the tau protein consisting of or within the amino acid sequence of SEQ ID NO: 1, wherein the antibody or antigen-binding fragment thereof binds paired helical filament (PHF)-tau, preferably human PHF-tau.

2. The isolated monoclonal antibody or antigen-binding fragment thereof of claim 1, wherein:
(a) the epitope of the tau protein comprises either one of phosphorylated S433 or phosphorylated S435 of the tau protein, but does not comprise phosphorylated S433 and phosphorylated S435;
(b) the epitope of the tau protein comprises one or more of phosphorylated T427, phosphorylated S433 and phosphorylated S435 of the tau protein, but does not comprise all of phosphorylated T427, phosphorylated S433 and phosphorylated S435;
(c) the epitope of the tau protein comprises one or more of phosphorylated T427 and phosphorylated S433 of the tau protein, but does not comprise phosphorylated S435, and does not comprise all of phosphorylated T427, phosphorylated S433 and phosphorylated S435; or
(d) the epitope of the tau protein comprises phosphorylated T427 of the tau protein, but does not comprise phosphorylated S433 or phosphorylated S435.

3. The isolated monoclonal antibody or antigen-binding fragment thereof of claim 1 comprising:
(a) immunoglobulin heavy chain HCDR1, HCDR2 and HCDR3 having the polypeptide sequences of SEQ ID NOs: 4, 5 and 6, respectively; and immunoglobulin light chain LCDR1, LCDR2 and LCDR3 having the polypeptide sequences of SEQ ID NOs: 7, 8 and 9, respectively;
(b) immunoglobulin heavy chain HCDR1, HCDR2 and HCDR3 having the polypeptide sequences of SEQ ID NOs: 14, 15 and 16, respectively; and immunoglobulin light chain LCDR1, LCDR2 and LCDR3 having the polypeptide sequences of SEQ ID NOs: 17, 18 and 19, respectively;
(c) immunoglobulin heavy chain HCDR1, HCDR2 and HCDR3 having the polypeptide sequences of SEQ ID NOs: 24, 25 and 26, respectively; and immunoglobulin light chain LCDR1, LCDR2 and LCDR3 having the polypeptide sequences of SEQ ID NOs: 27, 18 and 19, respectively;
(d) immunoglobulin heavy chain HCDR1, HCDR2 and HCDR3 having the polypeptide sequences of SEQ ID NOs: 32, 33 and 34, respectively; and immunoglobulin light chain LCDR1, LCDR2 and LCDR3 having the polypeptide sequences of SEQ ID NOs: 17, 18 and 35, respectively; or
(e) immunoglobulin heavy chain HCDR1, HCDR2 and HCDR3 having the polypeptide sequences of SEQ ID NOs: 40, 41 and 42, respectively; and immunoglobulin light chain LCDR1, LCDR2 and LCDR3 having the polypeptide sequences of SEQ ID NOs: 17, 18 and 43, respectively.

4. The isolated monoclonal antibody or antigen-binding fragment thereof of claim 1, comprising a heavy chain variable region having a polypeptide sequence at least 90% identical to SEQ ID NO: 2, 12, 22, 30 or 38, or a light chain variable region having a polypeptide sequence at least 90% identical to SEQ ID NO: 3, 13, 23, 31 or 39.

5. The isolated monoclonal antibody or antigen-binding fragment thereof of claim 1, comprising a heavy chain variable region having a polypeptide sequence of SEQ ID NO: 2, 12, 22, 30 or 38, or a light chain variable region having a polypeptide sequence of SEQ ID NO: 3, 13, 23, 31 or 39.

6. The isolated monoclonal antibody or antigen-binding fragment thereof of claim 1, comprising:
(a) a heavy chain variable region having the polypeptide sequence of SEQ ID NO: 2, and a light chain variable region having the polypeptide sequence of SEQ ID NO: 3;
(b) a heavy chain variable region having the polypeptide sequence of SEQ ID NO: 12, and a light chain variable region having the polypeptide sequence of SEQ ID NO: 13;
(c) a heavy chain variable region having the polypeptide sequence of SEQ ID NO: 22, and a light chain variable region having the polypeptide sequence of SEQ ID NO: 23;
(d) a heavy chain variable region having the polypeptide sequence of SEQ ID NO: 30, and a light chain variable region having the polypeptide sequence of SEQ ID NO: 31; or
(e) a heavy chain variable region having the polypeptide sequence of SEQ ID NO: 38, and a light chain variable region having the polypeptide sequence of SEQ ID NO: 39.

7. The isolated monoclonal antibody or antigen-binding fragment thereof of claim 1, comprising:
(a) a heavy chain having the polypeptide sequence of SEQ ID NO: 10, and a light chain having the polypeptide sequence of SEQ ID NO: 11;
(b) a heavy chain having the polypeptide sequence of SEQ ID NO: 20, and a light chain having the polypeptide sequence of SEQ ID NO: 21;
(c) a heavy chain having the polypeptide sequence of SEQ ID NO: 28, and a light chain having the polypeptide sequence of SEQ ID NO: 29;
(d) a heavy chain having the polypeptide sequence of SEQ ID NO: 36, and a light chain having the polypeptide sequence of SEQ ID NO: 37; or
(e) a heavy chain having the polypeptide sequence of SEQ ID NO: 44, and a light chain having the polypeptide sequence of SEQ ID NO: 45.

8. An isolated monoclonal antibody or antigen-binding fragment thereof, comprising:
(a) an immunoglobulin heavy chain HCDR1, HCDR2 and HCDR3 having the polypeptide sequences of SEQ ID NOs: 4, 5 and 6, respectively; and immunoglobulin light chain LCDR1, LCDR2 and LCDR3 having the polypeptide sequences of SEQ ID NOs: 7, 8 and 9, respectively;
(b) a heavy chain variable region having the polypeptide sequence of SEQ ID NO: 2, and a light chain variable region having the polypeptide sequence of SEQ ID NO: 3; or
(c) a heavy chain having the polypeptide sequence of SEQ ID NO: 10, and a light chain having the polypeptide sequence of SEQ ID NO: 11.

9. An isolated monoclonal antibody or antigen-binding fragment thereof, comprising:
(a) immunoglobulin heavy chain HCDR1, HCDR2 and HCDR3 having the polypeptide sequences of SEQ ID NOs: 14, 15 and 16, respectively; and immunoglobulin light chain LCDR1, LCDR2 and LCDR3 having the polypeptide sequences of SEQ ID NOs: 17, 18 and 19, respectively;
(b) a heavy chain variable region having the polypeptide sequence of SEQ ID NO: 12, and a light chain variable region having the polypeptide sequence of SEQ ID NO: 13; or
(c) a heavy chain having the polypeptide sequence of SEQ ID NO: 20, and a light chain having the polypeptide sequence of SEQ ID NO: 21.

10. An isolated nucleic acid encoding the isolated monoclonal antibody or antigen-binding fragment thereof of claim 1.

11. A vector comprising the isolated nucleic acid of claim 10.

12. A host cell comprising the isolated nucleic acid of claim 10.

13. A pharmaceutical composition comprising the isolated monoclonal antibody or antigen-binding fragment thereof of any one of claim 1 and a pharmaceutically acceptable carrier.

14. A method of blocking tau seeding in a subject in need thereof, comprising administering to the subject the pharmaceutical composition of claim 13.

15. A method of treating a tauopathy in a subject in need thereof, comprising administering to the subject the pharmaceutical composition of claim 13.

16. A method of reducing pathological tau aggregation or spreading of tauopathy in a subject in need thereof, comprising administering to the subject the pharmaceutical composition of claim 13.

17. The method of claim 15, wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

18. A method of producing the isolated monoclonal antibody or antigen-binding fragment thereof of claim 1, comprising culturing a cell comprising a nucleic acid encoding the monoclonal antibody or antigen-binding fragment thereof under conditions to produce the monoclonal antibody or antigen-binding fragment thereof, and recovering the monoclonal antibody or antigen-binding fragment thereof from the cell or cell culture.

19. A method of detecting the presence of PHF-tau in a biological sample from a subject, comprising contacting the biological sample with the monoclonal antibody or antigen-binding fragment thereof of claim 1, and detecting binding of the monoclonal antibody or antigen-binding fragment thereof to PHF-tau in the sample from the subject.

20. The method of claim 19, wherein the biological sample is a blood, serum, plasma, interstitial fluid, or cerebral spinal fluid sample.
